(19) United States
(12) Patent Application Publication
(10) Pub. No.: US 2021/0130316 A1
(43) Pub. Date: May 6, 2021

(54) MORPHINAN DERIVATIVES AND COMPOSITIONS COMPRISING THE SAME FOR TREATING AUTOIMMUNE, INFLAMMATION OR INFECTION RELATED DISORDERS

(71) Applicant: TAIWANJ PHARMACEUTICALS CO., LTD, Zhubei City, Hsinchu County (TW)

(72) Inventors: Syaulan S. YANG, Chesterfield, MO (US); Kuang-Yuan LEE, Hsinchu City (TW); Edwin SC WU, Cary, NC (US); Ming-Yu HSIAO, Shetou Township (TW); Hsiao-Chun WANG, Taipei City (TW); Meng-Hsien LIU, Toufen City (TW); Peter JS CHIU, Mill Creek, WA (US)

(73) Assignee: TAIWANJ PHARMACEUTICALS CO., LTD, Zhubei City, Hsinchu County (TW)

(21) Appl. No.: 16/473,745

(22) PCT Filed: Dec. 19, 2017
(86) PCT No.: PCT/US2017/067201

§ 371 Date: Jun. 26, 2019

Related U.S. Application Data
(60) Provisional application No. 62/442,945, filed on Jan. 5, 2017.

(51) Int. Cl. C07D 401/04; C07D 221/28

(52) U.S. Cl. CPC C07D 401/04; C07D 221/28

(57) ABSTRACT

Novel antagonists of toll-like receptor 4 (TLR-4) are provided. More specifically, the novel antagonists of TLR-4 are derived from morphinan. Further, use of said morphinan derivatives in the treatment of diseases and/or disorders mediated by TLR-4, such as autoimmune diseases, inflammation disease and infectious diseases, is provided. Pharmaceutical compositions including said morphinan derivatives are also provided.

### CROSS REFERENCE TO RELATED APPLICATION

This application claims the benefit of U.S. Provisional Application No. 62/442,945, filed on Jan. 5, 2017, the entirety of which is incorporated by reference herein.

### TECHNICAL FIELD

The present disclosure in general relates to the field of morphinans, more particularly to the derivatives of morphinans, and their use as toll-like receptor 4 (TLR-4) antagonists in the treatment of diseases and/or disorders resulted from the activation of TLR-4.

### BACKGROUND

Innate immune response is composed of inherent abilities to respond to microbial pathogens while toll-like receptors (TLRs) are key modulators of the innate immune system. TLRs are best known for their role in early host defense against infection and recently published evidence suggests that TLRs play a critical role in tissue homeostasis via regulating inflammation and related tissue repair in response to injury.

Toll-like receptor 4 (TLR-4) is a cell surface, type 1 transmembrane protein, present in macrophages such as the Kupffer cells of the liver. TLR4 plays an important role in recognizing and mediating macrophage activation and pro-inflammatory cytokine release along the immunological cascade. Activation of a TLR by its relevant ligand rapidly ignites a complex intracellular signaling cascade that ultimately results in upregulation of inflammatory genes and production of pro-inflammatory cytokines, interferons and recruitment of myeloid cells. It also stimulates expression, upon antigen presenting cells, of co-stimulatory molecules required to induce an adaptive immune response.

Although a robust TLR response is critical for survival and defense against invading pathogens, inappropriate signaling in response to alterations in the local microflora environment can be detrimental. Such ‘unhelpful TLR responses’ could result in various autoimmune diseases and inflammatory diseases.

Accordingly, development of TLR-4 antagonists in the treatment of diseases and/or disorders resulted from the activation of TLR-4 is needed.

### SUMMARY

The present disclosure is based on unexpected discovery that certain morphinan derivatives are potent antagonists of TLR-4, thus are useful as lead compounds for the development of medicaments for the treatment of diseases and/or disorders mediated by TLR-4, such as autoimmune diseases, inflammatory diseases and/or infectious diseases.

Accordingly, one aspect of the present disclosure is to provide a novel compound having the structure of Formula (I), or a pharmaceutically acceptable salt, solvate, hydrate, co-crystal, and stereoisomer thereof:

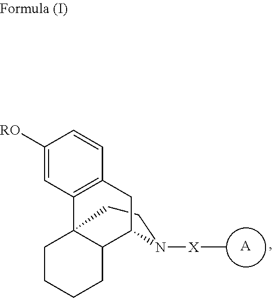

wherein:

R is H, C₁₋₂₀ alkyl, aryl, —(CH₂)ₙ—OR₁, or —(CH₂)—NR₁R₂;

X is nil, —SO₂—, or

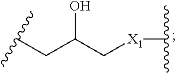

A is cycloalkyl, heterocycloalkyl, phenyl, aryl or heteroaryl;

n is an integral between 1 to 10,

R₁ and R₂ are independently H, C₁₋₂₀ alkyl, C₁₋₂₀ alkoxy, phenyl, or aryl;

X₁ is nil, S or O;

each C₁₋₂₀ alkyl and C₁₋₂₀ alkoxy is optionally substituted with at least one substituent selected from the group consisting of halogen, hydroxy, alkoxy, and phenyl;

each cycolalkyl, heterocycloalkyl, phenyl, aryl and heteroaryl is optionally substituted with at least one substituent selected from the group consisting of halogen, C₁₋₂₀ alkyl, hydroxyl, alkoxy, haloalkyl, haloalkoxy, nitro, —NR₃R₄, —(CO)R₁ and —(CO)OR₁; and

R₃ and R₄ are independently H, C₁₋₂₀ alkyl, phenyl, aryl, —(CO)R₁ or —(CO)OR₁.

In certain embodiments, the compound of formula (I) can be the compound of formula (II), or a pharmaceutically acceptable salt, solvate, hydrate, co-crystal, and stereoisomer thereof:

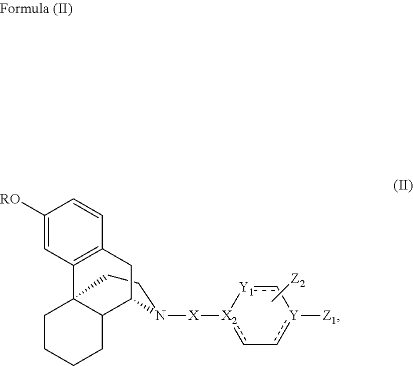

wherein:

R is independently H, C₁₋₂₀alkyl, aryl, —(CH₂)ₙ—OR₁, or —(CH₂)ₙ—NR₁R₂;

X is nil, —SO₂—, or

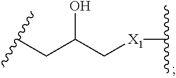

[symbol] is a single or double bond;

X₁ is nil, S or O;

X₂ is N or C;

Y is N, C or O;

Y₁ is C or N;

n is an integral between 1 to 10;

Z₁ and Z₂ are independently H, halogen, C₁₋₂₀ alkyl, —NR₁R₂, nitro, —OR₁, —NH(CO)R₁, and —N(CO)OR₁;

R₁ and R₂ are independently H, halogen, C₁₋₂₀ alkyl, C₁₋₂₀ alkoxy, phenyl, or aryl;

each C₁₋₂₀ alkyl and C₁₋₂₀ alkoxy is optionally substituted with at least one substituent selected from the group consisting of halogen, hydroxy, alkoxy, and phenyl;

each phenyl and aryl is optionally substituted with at least one substituent selected from the group consisting of halogen, C₁₋₂₀ alkyl, hydroxyl, alkoxy, haloalkoxy, nitro, —NR₃R₄, —(CO)R₁ and —(CO)OR₁; and

R₃ and R₄ are independently H, C₁₋₂₀ alkyl, phenyl, aryl, —(CO)R₁ or —(CO)OR₁.

In certain embodiments, the compound of Formula (I) can be the compound of Formula (III), or a pharmaceutically acceptable salt, solvate, hydrate, co-crystal, and stereoisomer thereof:

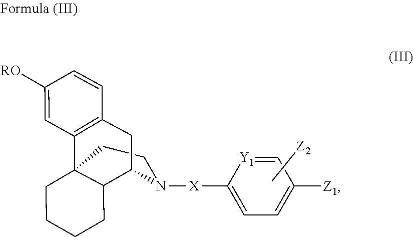

wherein:

R is independently H, or C₁₋₂₀ alkyl;

X is nil or —SO₂—;

Y₁ is C or N;

Z₁ and Z₂ are independently H, —NR₁R₂, nitro, —OR, —R₅, or —NH(CO)R₅; and R₁ and R₂ are independently H, halogen, C₁₋₂₀ alkyl, C₁₋₂₀ alkoxy, phenyl, or aryl; R₅ is H, halogen, C₁₋₂₀ alkyl or C₁₋₂₀ alkoxy optionally substituted with halogen or phenyl.

In one embodiment, in Formula (III), R is methyl or H, X is nil, Y₁ is C, and Z₁ is nitro or amino, and Z₂ is H or amino.

In another embodiment, in Formula (III), R is H, X is nil, Y₁ is C, Z, is amino, and Z₂ is R₅, R₅ is fluorine, methyl, or trifluoromethyl.

In further embodiment, in Formula (III), R is H, X is nil, Y₁ is C, and Z₁ is —NR₁R₂, each of R, R₂ is H or isopropyl, and Z₂ is H.

In another embodiment, in Formula (III), R is methyl, X is nil, Y₁ is C, and Z₁ is —NH(CO)R₅, Z₂ is H, and R₅ is ethoxy.

In still another embodiment, in Formula (III), R is methyl, X is nil, Y₁ is C, and Z₁ is —NH(CO)R₅, Z₂ is H, and R₅ is propyl substituted with a phenyl.

In another further embodiment, in Formula (III), R is H, X is —SO₂—, Y₁ is C, Z₁ is —NH(CO)R₅, Z₂ is H, and R₅ is methyl.

In still another embodiment, in Formula (III), R is H, X is —SO₂—, Y₁ is C, Z₁ is —NR₁R₂, each of R₁, R₂ is H, and Z₂ is H.

In further embodiment, in Formula (III), R is methyl, X is —SO₂—, Y₁ is C, and Z₁ is nitro, and Z₂ is H.

In yet another embodiment, in Formula (III), R is methyl, X is —SO₂—, Y₁ is C, and Z₁ is amino, and Z₂ is H.

In still another embodiment, R is H, X is nil, Y₁ is N, and Z₁ is nitro, and Z₂ is H.

In further embodiment, R is H, X is nil, Y₁ is N, and Z₁ is amino, and Z₂ is H.

In certain embodiments, the compound of Formula (I) can be the compound of Formula (IV), or a pharmaceutically acceptable salt, solvate, hydrate, co-crystal, and stereoisomer thereof:

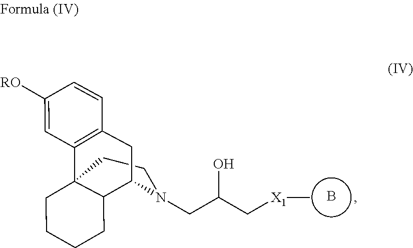

wherein:

R is independently H, or C₁₋₂₀ alkyl

X₁ is nil, S or O;

B is phenyl or heterocycloalkyl optionally substituted with halogen, C₁₋₂₀ alkyl, alkoxy, haloalkoxy, amino or nitro.

In one embodiment, in Formula (IV), R is methyl, X₁ is nil, and B is a 6-membered heterocycloalkyl.

In another embodiment, in Formula (IV), R is methyl, X₁ is S, and B is phenyl.

In a further embodiment, in Formula (IV), R is methyl, X₁ is O and B is phenyl.

A further aspect of the present disclosure is to provide a pharmaceutical composition for the treatment of a subject having or suspected of having a disease and/or disorder mediated by TLR-4. The disease and/or disorder mediated by TLR-4 may be an autoimmune disease, an inflammatory disease or an infectious disease. The pharmacological composition comprises a therapeutically effective amount of the compound of Formula (I) to (IV); and a pharmaceutically acceptable carrier.

The compound of Formula (I) to (IV) is present at a level of about 0.1% to 99% by weight, based on the total weight of the pharmaceutical composition. In some embodiments, the compound of Formula (I) to (IV) is present at a level of at least 1% by weight, based on the total weight of the pharmaceutical composition. In certain embodiments, the compound of Formula (I) to (IV) is present at a level of at least 5% by weight, based on the total weight of the pharmaceutical composition. In still other embodiments, the compound of Formula (I) to (IV) is present at a level of at least 10% by weight, based on the total weight of the pharmaceutical composition. In still yet other embodiments, the compound of Formula (I) to (IV) is present at a level of at least 25% by weight, based on the total weight of the pharmaceutical composition.

According to some preferred embodiments, the pharmaceutical composition further comprises an immunosuppressant, an anti-inflammation agent or anti-infectious agent. In some examples, the immunosuppressant may be a corticosteroid, a calcineurin inhibitor, a mammalian target of rapamycin (mTOR) inhibitor, an Inosine monophosphate dehydrogenase (IMPDH) inhibitor, a therapeutic protein, or a monoclonal antibody. In some examples, the anti-inflammation agent may be a cyclooxygenase (COX) inhibitor, particularly a COX-2 inhibitor. In some examples, the anti-infectious agent may be an anti-biotic that inhibits the growth of Gram-negative or Gram-positive bacteria.

The present disclosure also encompasses a method for the treatment of a subject having a disease and/or disorder mediated by the activation of TLR-4. The method comprises the step of administering the present pharmaceutical composition to the subject, so as to ameliorate, mitigate and/or prevent the symptoms of the disease and/or disorder mediated by TLR-4. The disease and/or disorder mediated by TLR-4 may be an autoimmune disease, an inflammatory disease or an infectious disease.

Examples of the autoimmune disease treatable by the present method include, but are not limited to, multiple sclerosis, psoriasis, systemic lupus erythemotosis (SLE), Type I diabetes mellitus, and Wegener's granulomatosis.

Examples of the inflammatory disease treatable by the present method include, but are not limited to, asthma, allergic rhinitis, acute and chronic liver diseases, atherosclerosis, cancer, Crohn's disease, hypersensitivity lung disease, irritable bowel syndrome (IBS), inflammatory dermatoses, Sjogren's syndrome, systemic inflammatory response syndrome (SIRS), and ulcerative colitis.

Examples of the infectious disease treatable by the present method include, but are not limited to, bacterial, fungal and viral infections.

According to some preferred embodiments, the method further includes the step of administering to the subject another agent that is known to improve the symptoms of the disease and/or disorder mediated by TLR-4, such as an immunosuppressant, an anti-inflammation agent, or an anti-infectious agent before, together with, or after the administration of the present pharmaceutical composition.

The details of one or more embodiments of this disclosure are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed

A detailed description is given in the following embodiments.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### 1. Definitions

When a range of values is listed, it is intended to encompass each value and sub-range within the range. For example “C₁₋₄” is intended to encompass, C₁, C₂, C₃, C₄, C₁₋₄, C₁₋₃, C₁₋₂, C₂₋₄, C₂₋₃, and C₃₋₄.

Unless otherwise indicated, the term “alkyl” means a straight chain, branched and/or cyclic (“cycloalkyl”) hydrocarbon having from 1 to 20 (e.g., 1 to 10, 1 to 9, 1 to 8, 1 to 7, 1 to 6, 1 to 5, 1 to 4, 1 to3, to 2, or 1) carbon atoms. Alkyl moieties having from 1 to 4 carbons (C₁₋₄ alkyl) are referred to as “lower alkyl.” Examples of alkyl groups include methyl, ethyl, propyl, isopropyl, n-butyl, t-butyl, isobutyl, 2-isopropyl-3-methyl butyl, pentyl, pentan-2-yl, hexyl, isohexyl, heptyl, heptan-2-yl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, and cyclohexyl. Unless otherwise specified, each instance of an alkyl group is independently optionally substituted, i.e., unsubstituted (an “unsubstituted alkyl”) or substituted (a “substituted alkyl”) with one or more substituents. In certain embodiments, the alkyl group is substituted C₂₋₁₀ alkyl. In some embodiments, cycloalkyl is a monocyclic, saturated carbocyclyl group having from 3 to 6 ring carbon atoms (“C₃₋₆ cycloalkyl”). In some embodiments, a cycloalkyl group has 5 to 6 ring carbon atoms (“C₅₋₆ cycloalkyl”). Examples of C₅₋₆ cycloalkyl groups include cyclopentyl (C₅) and cyclohexyl (C₆). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocycloalkyl” refers to a radical of a 3- to 10-membered non-aromatic ring system having ring carbon atoms and 1 to 4 ring heteroatoms, wherein each heteroatom is independently selected from nitrogen, oxygen, sulfur, phosphorus, and silicon (“3-10 membered heterocycloalkyl”). In heterocyclyl groups that contain one or more nitrogen atoms, the point of attachment can be a carbon or nitrogen atom, as valency permits. Unless otherwise specified, each instance of heterocycloalkyl is independently optionally substituted, i.e., unsubstituted (an “unsubstituted heterocycloalkyl”) or substituted (a “substituted heterocycloalkyl”) with one or more substituents. In some embodiments, a heterocyclyl group is a 5-8 membered non-aromatic ring system having ring carbon atoms and 1-4 ring heteroatoms, wherein each heteroatom is independently selected from nitrogen, oxygen, and sulfur. In some embodiments, a heterocycloalkyl group is a 5-6 membered non-aromatic ring system having ring carbon atoms and 1-4 ring heteroatoms, wherein each heteroatom is independently selected from nitrogen, oxygen, and sulfur. In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocycloalkyl has one ring heteroatom selected from nitrogen, oxygen, and sulfur. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl.

Unless otherwise indicated, the term “aryl” means an aromatic ring or a partially aromatic ring system composed of carbon and hydrogen atoms. An aryl moiety may comprise multiple rings bound or fused together. Examples of aryl moieties include naphthyl, and phenyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is a substituted phenyl.

Unless otherwise indicated, the term “heteroaryl” means an aryl moiety wherein at least one of its carbon atoms has been replaced with a heteroatom (e.g., N, O or S). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl. Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl.

Unless otherwise indicated, the term “alkylaryl” or “alkyl-aryl” means an alkyl moiety bound to an aryl moiety.

Unless otherwise indicated, the term “alkoxy” means an —O-alkyl group. Examples of alkoxy groups include, but are not limited to, —OCH₃, —OCH₂CH₃, —O(CH₂)₂CH₃, —O(CH₂)₃CH₃, —O(CH₂)₄CH₃, and —O(CH₂)₅CH₃. The term “lower alkoxy” refers to —O-(lower alkyl), such as —OCH₃ and —OCH₂CH₃.

Unless otherwise indicated, the terms “halogen” and “halo” encompass fluoro, chloro, bromo, and iodo.

The term “amino” refers to a moiety of the formula: —N(R)₂, wherein each instance of R is independently a substituent described herein, or two instances of R are connected to form substituted or unsubstituted heterocyclyl. In certain embodiments, the amino is unsubstituted amino (i.e., —NH₂). In certain embodiments, the amino is a substituted amino group, wherein at least one instance of R is not hydrogen.

Unless otherwise indicated, the term “substituted,” when used to describe a chemical structure or moiety, refers to a derivative of that structure or moiety wherein one or more of its hydrogen atoms is substituted with an atom, chemical moiety or functional group such as, but not limited to, —OH, —CHO, alkoxy, alkanoyloxy (e.g., —OAc), alkenyl, alkyl (e.g., methyl, ethyl, propyl, t-butyl), aryl, aryloxy, halo, or haloalkyl (e.g., —CCl₃, —CF₃, —C(CF₃)₃).

In a particular embodiment, the term “substituted,” when used to describe a chemical structure or moiety, refers to a derivative of that structure or moiety wherein one or more of its hydrogen atoms is substituted with one or more of: alkoxy, alkanoyloxy, alkyl, aryl, halo, haloalkyl, or hydroxyl.

Unless otherwise indicated, one or more adjectives immediately preceding a series of nouns is to be construed as applying to each of the nouns. For example, the phrase “optionally substituted alky, cycloalkyl, heterocycloalkyl, aryl, or heteroaryl” has the same meaning as “optionally substituted alky, optionally substituted cycloalkyl, optionally substituted heterocycloalkyl, optionally substituted aryl, or optionally substituted heteroaryl.”

The present disclosure is not intended to be limited in any manner by the above exemplary listing of substituents.

The term “solvate” refers to forms of the compound that are associated with a solvent, usually by a solvolysis reaction. This physical association may include hydrogen bonding. Conventional solvents include water, methanol, ethanol, acetic acid, dimethyl sulfoxide (DMSO), tetrahydrofuran (THF), diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R.0.5 H₂O)), and polyhydrates (x is a number greater than 1, e.g., dihydrates (R.2 H₂O) and hexahydrates (R.6 H₂O)).

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

It should also be noted that if the stereochemistry of a structure or a portion of a structure is not indicated with, for example, bold or dashed lines, the structure or the portion of the structure is to be interpreted as encompassing all stereoisomers of it. Similarly, names of compounds having one or more chiral centers that do not specify the stereochemistry of those centers encompass pure stereoisomers and mixtures thereof. Moreover, any atom shown in a drawing with unsatisfied valences is assumed to be attached to enough hydrogen atoms to satisfy the valences.

Unless otherwise indicated, “an effective amount” of a compound is an amount sufficient to provide a therapeutic benefit in the treatment or management of a disease or condition, or to delay or minimize one or more symptoms associated with the disease or condition. A therapeutically effective amount of a compound is an amount of therapeutic agent, alone or in combination with other therapies, which provides a therapeutic benefit in the treatment or management of the disease or condition. The term “effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder, or one or more of its symptoms, or retards or slows the progression of the disease or disorder.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

The term “pharmaceutically acceptable carrier” refers to a carrier, whether diluent or excipient, that is compatible with the other ingredients of a formulation and not deleterious to the recipient thereof.

The terms “administration of a composition” or “administering a composition” is defined to include an act of providing a compound or pharmaceutical composition of the present invention to the subject in need of treatment.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The singular forms “a”, “and”, and “the” are used herein to include plural referents unless the context clearly dictates otherwise.

### 2. Novel Compounds

The compounds as described herein can have the structure of Formula (I), or a pharmaceutically acceptable salt, solvate, hydrate, co-crystal, and stereoisomer thereof:

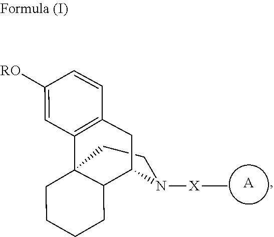

In the Formula (I), R may be H, C₁₋₂ alkyl, aryl, —(CH₂)ₙ—OR₁, or —(CH₂)—NR₁R₂, in which n is an integral between 1 to 10, and R₁ and R₂ may be independently H, C₁₋₂₀ alkyl, C₁₋₂₀ alkoxy, phenyl, or aryl. Optionally, each C₁₋₂₀ alkyl and C₁₋₂₀ alkoxy is substituted with at least one substituent selected from the group consisting of halogen, hydroxy, alkoxy, and phenyl.

In certain embodiments, R is C₁₋₂₀ alkyl, such as methyl, ethyl, propy isopropyl, n-butyl, t-butyl, isobutyl, 2-isopropyl-3-methyl butyl, pentyl, pentan-2-yl, hexyl, isohexyl, heptyl, heptan-2-yl, 4,4-dimethylpentyl, octyl, 2,2,4-trimethylpentyl, nonyl, decyl, undecyl and dodecyl. In some preferred examples, R is methyl. In other examples, R is H.

In the Formula (I), X may be nil, —SO₂—, or

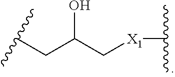

Alternatively or additionally, X₁ may be nil, S or O.

In the Formula (I), A may be selected from the group consisting of cycloalkyl, heterocycloalkyl, phenyl, aryl, and heteroaryl, and each cycolalkyl, heterocycloalkyl, phenyl, aryl and heteroaryl may be optionally substituted with at least one substituent selected from the group consisting of halogen, C₁₋₂₀ alkyl, hydroxyl, alkoxy, haloalkoxy, haloalkyl, nitro, —NR₃R₄, —(CO)R₁ and —(CO)OR₁. Alternatively or additionally, each R₃ and R₄ may be independently H, C₁₋₂₀ alkyl, phenyl, aryl, —(CO)R₁ or —(CO)OR₁.

In certain embodiments, X is nil, and A is phenyl optionally substituted with at least one substituent selected from the group consisting of halogen, C₁₋₂₀ alkyl, alkoxy, haloalkyl, haloalkoxy, nitro, —NR₃R₄, —(CO)R₁ and —(CO)OR₁. In some examples, A is phenyl substituted with a nitro group. In other examples, A is phenyl substituted with —NR₃R₄, where both R₃ and R₄ are H or C₁₋₂₀ alkyl. In further examples, A is phenyl substituted with a nitro group and an amino group. In still further examples, A is phenyl substituted with —NR₃R₄, in which R₃ and R₄ may be independently H, and —(CO)R₁, in which R₁ is ethoxy. In In still further examples, A is phenyl substituted with —NR₃R₄, in which R₃ and R₄ may be independently H, and —(CO)OR₁, in which R₁ is propyl substituted with a phenyl group. In still further examples, A is pyridinyl optionally substituted with at least one substituent selected from the group consisting of halogen, C₁₋₂₀ alkyl, haloalkyl, nitro, and amino group.

In certain embodiments, X is —SO₂—, and A is a phenyl optionally substituted with at least one substituent selected from the group consisting of halogen, C₁₋₂₀ alkyl, hydroxyl, alkoxy, haloalkoxy, nitro, —NR₃R₄, —(CO)R₁ and —(CO)OR₁. In some examples, A is phenyl substituted with a nitro. In other examples, A is phenyl substituted with a —NR₃R₄, where both R₃ and R₄ are H.

In certain embodiments, X is

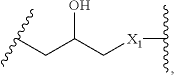

in which X₁ is S, and A is a phenyl.

In other embodiments, X is

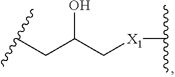

in which X₁ is O, and A is a phenyl.

In further embodiments, X is

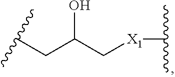

in which X₁ is nil, and A is a heterocycloalkyl, such as morpholinyl.

In certain embodiments, the compound of Formula (I) can be of Formula (II), or a pharmaceutically acceptable salt, solvate, hydrate, co-crystal, and stereoisomer thereof:

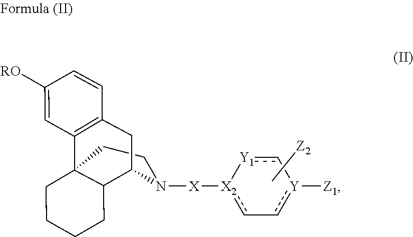

wherein:

R is independently H, C₁₋₂₀alkyl, aryl, —(CH₂)ₙ—OR₁, or —(CH₂)ₙ—NR₁R₂;

X is nil, —SO₂—, or

[symbol] is a single or double bond;

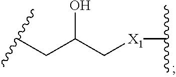

X₁ is nil, S or O;

X₂ is N or C;

Y is N, C or O;

Y₁ is C or N;

n is an integral between 1 to 10;

Z₁ and Z₂ are independently H, C₁₋₂₀ alkyl, amino, nitro, —ORI, —CRI, —NH(CO)R₁, and —N(CO)OR₁;

R₁ and R₂ are independently H, halogen, C₁₋₂₀ alkyl, C₁₋₂₀ alkoxy, phenyl, or aryl;

each C₁₋₂₀ alkyl and Ca-20 alkoxy is optionally substituted with at least one substituent selected from the group consisting of halogen, hydroxy, alkoxy, and phenyl;

each phenyl and aryl is optionally substituted with at least one substituent selected from the group consisting of halogen, C₁₋₂₀ alkyl, hydroxyl, alkoxy, haloalkoxy, nitro, —NR₃R₄, —(CO)R₁ and —(CO)ORI; and

R₃ and R₄ are independently H, C₁₋₂₀ alkyl, phenyl, aryl, —(CO)R₁ or —(CO)OR₁.

In certain embodiments, the compound of Formula (I) can be of Formula (III), or a pharmaceutically acceptable salt, solvate, hydrate, co-crystal, and stereoisomer thereof:

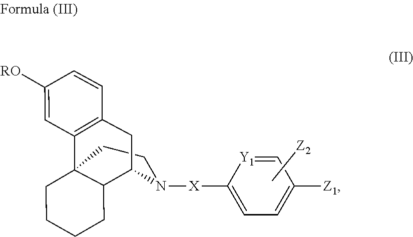

wherein:

R is independently H, or C₁₋₂₀ alkyl;

X is nil or —SO₂—;

Y₁ is C or N;

Z₁ and Z₂ are independently H, amino, nitro, —OR, —CR₅, or —NH(CO)R₅; and

R₅ is H, halogen, C₁₋₂₀ alkyl or C₁₋₂₀ alkoxy optionally substituted with halogen or phenyl.

According to one preferred embodiment, in the Formula (III), R is H, X is nil, Z₁ is amino, and Z₂ is H.

According to another preferred embodiment, in the Formula (III), R is methyl, X is SO₂, Z₂ is H, and Z₂ is amino.

In further embodiments, the compound of Formula (I) can be of Formula (IV), or a pharmaceutically acceptable salt, solvate, hydrate, co-crystal, and stereoisomer thereof:

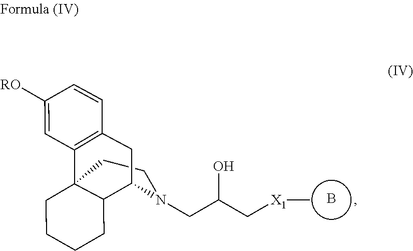

wherein:

R is independently H, or C₁₋₂₀ alkyl

X₁ is nil, S or O;

B is phenyl or heterocycloalkyl optionally substituted with halogen, C₁₋₂₀ alkyl, alkoxy, haloalkoxy, amino or nitro.

According to one preferred embodiment, in the Formula (IV), R is methyl, X₁ is S, and B is phenyl.

According to another preferred embodiment, in the Formula (IV), R is methyl, X₁ is O, and B is phenyl.

Each compounds of the invention contain one or more stereocenters, thus can exist as racemic mixtures of enantiomers or mixtures of diastereomers. This invention thus encompasses stereomerically pure forms of such compounds, as well as mixtures of those forms. Stereoisomers may be asymmetrically synthesized or resolved using standard techniques such as crystallization, chromatography, and the use of a resolving agent. One preferred way of separating enantiomers from a racemic mixture is by use of preparative high performance liquid chromatography (HPLC). Alternatively, the racemic may be separated into its enantiomers by reacting with an optically active form of a resolving agent in the presence of a solvent. Depending on the optical form of the resolving agent, one of the two enantiomers is separated out as an insoluble salt with high yield and high optical purity, while the opposite enantiomer remains in the solution. The present invention thus further encompasses stereoisomeric mixtures of compounds disclosed herein. It also encompasses configurational isomers of compounds disclosed herein (e.g., cis and trans isomers, whether or not involving double bonds), either in admixture or in pure or substantially pure form.

In certain embodiments, compounds of the invention are the compounds described herein, and pharmaceutically acceptable salts, solvates, hydrates, co-crystals, and stereoisomers. In certain embodiments, compounds of the invention are the compounds of any one of Formulae (I) to (IV), and pharmaceutically acceptable salts, solvates, hydrates, co-crystals, and stereoisomers. In certain embodiments, compounds of the invention are the compounds of any one of Formulae (I) to (IV), and pharmaceutically acceptable salts thereof.

According to preferred embodiments of the present disclosure, the compound of Formula (I) may suppress the NF-κB induced secretion of secreted alkaline phosphatase (SEAP), as well as lipopolysaccharide (LPS) induced TNF-α release, both mechanisms are respectively linked to the activation of toll-like receptor 4 (TLR-4). Accordingly, the compound of Formula (I) acts as an antagonist of TLR-4, thus may be useful as a lead compound for the development of a medicament suitable for the treatment of diseases and/or disorders mediated by TLR-4.

### 3. Methods of Synthesis

Any of the compounds described herein can be prepared by routine methods known in the art. In some embodiments, a compound as described herein (e.g., a compound of Formula (I)) is synthesized by reacting a morphinan of Formula (V) with a suitable compound (e.g., 4-bromonitrobenzene or 2-(phenoxymethyl) oxirane):

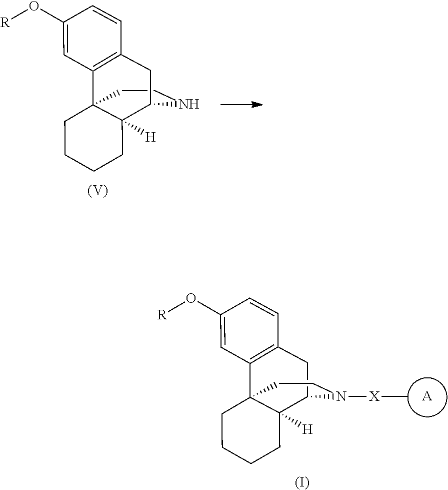

wherein:

R is H, C₁₋₂₀ alkyl, aryl, —(CH₂)ₙ—OR₁, or —(CH₂)ₙ—NR₁R₂;

X is nil, —SO₂—, or

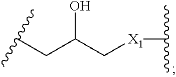

A is cycloalkyl, heterocycloalkyl, phenyl, aryl or heteroaryl;

n is an integral between 1 to 10;

R₁ and R₂ are independently H, C₁₋₂₀ alkyl, C₁₋₂₀ alkoxy, phenyl, or aryl;

X₁ is nil, S or O;

each C₁₋₂₀ alkyl and C₁₋₂₀ alkoxy is optionally substituted with at least one substituent selected from the group consisting of halogen, hydroxy, alkoxy, and phenyl;

each cycolalkyl, heterocycloalkyl, phenyl, aryl and heteroaryl is optionally substituted with at least one substituent selected from the group consisting of H, halogen, hydroxyl, C₁₋₂₀ alkyl, alkoxy, haloalkyl, haloalkoxy, nitro, —NR₃R₄, —(CO)R₁ and —(CO)OR₁; and

R₃ and R₄ are independently H, C₁₋₂₀ alkyl, phenyl, aryl, —(CO)R₁ or —(CO)ORI;

A Lewis acid can be used as a reaction promotor, which is selected from a group consisting of lithium, sodium, magnesium, calcium, zinc, aluminum, boron, indium, scandium, ytterbium, cerium, silicon, tin, titanium, zirconium, vanadium, iron, and cobalt salts with the counter anion selected from a group consisting of fluoride, chloride, bromide, iodide, hydroxide, methoxide, ethoxide, isoproproxide, tert-butoxide, acetate, oxalate, acetylacetonate, nitrate, phosphate, sulfate, bisulfate, and sulfonate. According to embodiments of the present disclosure, sodium tert-butoxide is used as the reaction promoter.

The reaction (e.g., the step of contacting) can be conducted in a hydrocarbon, chlorinated, and alcoholic solvent, or a mixture thereof (e.g., their co-solvent system in varied ratios). The hydrocarbon solvent comprises an acyclic, cyclic, or aromatic solvent selected from the group consisting of n-hexane, cyclohexane, benzene, toluene, and xylene.

Additionally or optionally, to assist the chain transfer reaction, the reaction promotor described above is mixed with a phosphonium based ionic liquid having a formula of [PHR₃]⁺X⁻, in which R is an alkly, and X is a counter anion. According to preferred embodiments, sodium tert-butoxide (i.e., the reaction promoter) is mixed with tri-tert-butylphosphonium tetrafluoroborate (i.e., a phosphonium based ionic liquid), and the reaction is conducted in toluene.

According to some embodiments, compounds of Formula (III) is prepared by reacting a morphinan of Formula (V) with a suitable aryl compound:

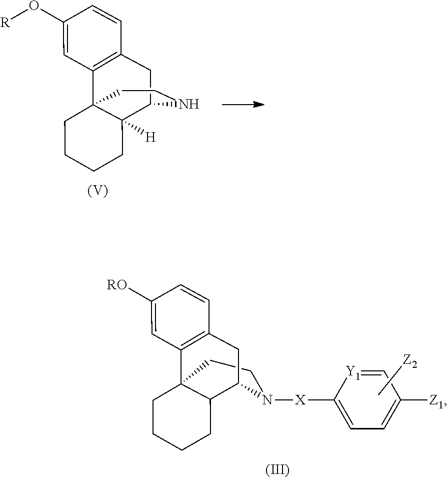

wherein:

R is independently H, or C₂₀ alkyl;

X is nil or —SO₂—;

Y₁ is C or N;

Z₁ and Z₂ are independently H, C₁₋₂₀ alkyl, —CR₅, amino, nitro, or —NH(CO)R₅; and

R₅ is halogen, C₁₋₂₀ alkyl, or C₁₋₂₀ alkoxy optionally substituted with halogen or phenyl.

Depending on the desired side chain to be introduced into the compound of Formula (III), an aryl compound having suitable substituent(s) may be reacted with the compound of Formula (V). For example, 4-bromonitobeneze is mixed with 3-methoxymorphinan (compound 1) in toluene, which contains sodium tert-butoxide as the reaction promoter and tri-tert-butylphosphonium tetrafluoroborate as the phosphonium based ionic liquid, allow the reaction to proceed until a product (i.e., compound 2) is produced. Let compound 2 react with hydrogen bromide, thereby produces compound 3; or palladium/carbon under hydrogen gas to produce compound 6. Alternatively, 3-methoxymorphinan (compound 1) is reacted with 5-bromo-2-nitroaniline or 4-nitrobenezesulfonyl chloride in a similar manner to produce compound 5 or compound 9. Additionally, compounds 3 and 9 may be further subject to hydrogenation to produce compounds 4 and 10, respectively. With similar methods above, by using 1-bromo-2-fluoro-4-nitrobenzene, 1-bromo-3-methyl-4-nitrobenzene, 1-bromo-2-trifluoromethyl-4-nitrobenzene, 2-bromo-5-nitro-pyridine, etc., compound 21, 22, 23, 24, and 25 are produced, respectively. Alternatively, 3-methoxymorphinan (compound 1) is reacted with HBr to produce compound 14, which is further reacted with N-acetylsulfonyl chloride to produce compound 15. By reacting Compound 15 to 12N hydrochloric acid at high temperature, compound 16 is then produced. With the same method for producing compound 3 and 4, using compound 5 as starting material, compound 17 and 18 are produced, respectively. Alternatively, compound 4 is reacted with acetone, acetic acid and NaHB(OAc)₃ to produce compound 19 and 20, respectively.

Alternatively, instead of 3-methoxymorphinan (compound 1), morphinan is used as the starting material in the described reaction, (that is, by first reacting with 4-bromonitobeneze, followed by hydrogenation) to produce compound 6, which may then respectively reacted with diisopropylethyl acetate (DIPEA) and 4-phenylbutanoic acid, to produce compounds 7 and 8.

By this approach, compounds of Formula (III) of this invention may be synthesized, respective side chains of each compounds of Formula (III) are summarized in Table 1:

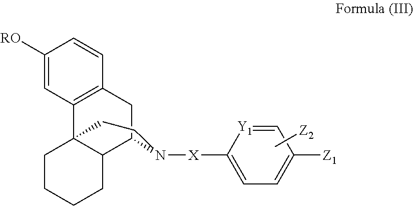

TABLE 1
Side chains of each compounds of Formula (III)

| Com- | | | | | |
|---|---|---|---|---|---|
| pound | | | | | |
| No. | R | X | Y₁ | Z₁ | Z₂ |
| 3 | H | nil | C | —NO₂ | H |
| 4 | H | nil | C | —NH₂ | H |
| 5 | CH₃ | nil | C | —NO₂ | —NH₂ |
| | | | | | (meta to X) |
| 6 | CH₃ | nil | C | —NH₂ | H |
| 7 | CH₃ | nil | C | —NH(CO)OC₂H₅ | H |
| 8 | CH₃ | nil | C | —NH(CO)C₃H₆(Phenyl) | H |
| 9 | CH₃ | —SO₂ | C | —NO₂ | H |
| 10 | CH₃ | —SO₂ | C | —NH₂ | H |
| 15 | H | —SO₂ | C | —NH(CO)CH₃ | H |
| 16 | H | —SO₂ | C | —NH₂ | H |
| 17 | H | nil | C | —NO₂ | —NH₂ |
| | | | | | (meta to X) |
| 18 | H | nil | C | —NH₂ | —NH₂ |
| | | | | | (meta to X) |
| 19 | H | nil | C | —N(isopropyl)₂ | H |
| 20 | H | nil | C | —NH(isopropyl) | H |
| 21 | H | nil | C | —NH₂ | —F |
| | | | | | (ortho to X) |
| 22 | H | nil | C | —NH₂ | —CH₃ |
| | | | | | (meta to X) |
| 23 | H | nil | C | —NH₂ | —CF₃ |
| | | | | | (ortho to X) |
| 24 | H | nil | N | —NO₂ | H |
| 25 | H | nil | N | —NH₂ | H |

According to other embodiments, compounds of Formula (IV) is prepared by reacting a morphinan of Formula (V) with an ethylene oxide derivative of formula

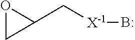

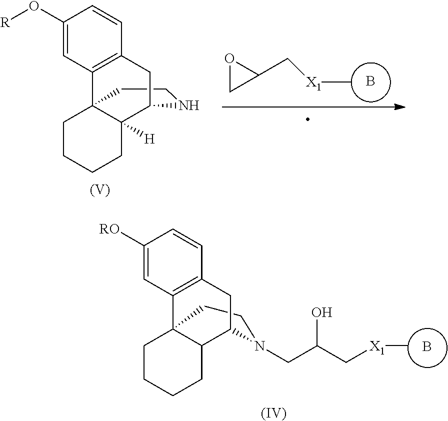

wherein:

R is independently H, or C₁₋₂₀ alkyl;

X₁ is nil, S or O;

B is phenyl or heterocycloalkyl optionally substituted with halogen, C₁₋₂₀ alkyl, alkoxy, haloalkoxy, amino or nitro.

Depending on the desired side chain to be introduced into the compound of Formula (IV), a compound of formula

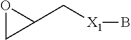

having suitable substituent(s) is reacted with the compound of Formula (V). For example, 2-(phenoxymethyl)oxirane is reacted with 3-methoxymorphinan (compound 1) to produce compound 11. Alternatively, 2-((phenylthio)methyl)oxirane and 4-(oxiran-2-yl)methyl)morpholine are respectively reacted with 3-methoxymorphinan (compound 1) to produce compounds 12 and 13.

By this approach, compounds of Formula (IV) of this invention may be synthesized, respective side chains of each compounds of Formula (IV) are summarized in Table 2:

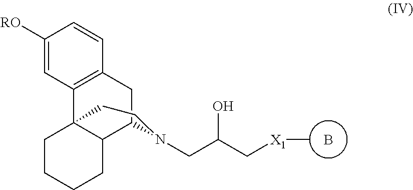

TABLE 2
Side chains of each compounds of Formula (IV)

| Compound No. | R | X₁ | B |
|---|---|---|---|
| 11 | CH₃ | O | phenyl |
| 12 | CH₃ | S | phenyl |
| 13 | CH₃ | nil | 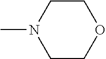 |

### 4. Pharmaceutical Formulation

This invention encompasses pharmaceutical compositions for the treatment of a disease and/or a disorder mediated by TLR-4. The pharmaceutical composition comprises a therapeutically effective amount of a compound of Formula (I) to (IV) of the present invention.

The compound of Formula (I) to (IV) is present at a level of about 0.1% to 99% by weight, based on the total weight of the pharmaceutical composition. In some embodiments, the compound of Formula (I) to (IV) is present at a level of at least 1% by weight, based on the total weight of the pharmaceutical composition. In certain embodiments, the compound of Formula (I) to (IV) is present at a level of at least 5% by weight, based on the total weight of the pharmaceutical composition. In still other embodiments, the compound of Formula (I) to (IV) is present at a level of at least 10% by weight, based on the total weight of the pharmaceutical composition. In still yet other embodiments, the compound of Formula (I) to (IV) is present at a level of at least 25% by weight, based on the total weight of the pharmaceutical composition.

According to embodiments of the present disclosure, the disease and/or disorder mediated by TLR-4 may be an autoimmune disease, an inflammatory disease or an infectious disease. Examples of the autoimmune disease treatable by the present pharmaceutical composition include, but are not limited to, multiple sclerosis, psoriasis, systemic lupus erythemotosis (SLE), Type I diabetes mellitus, and Wegener's granulomatosis. Examples of the inflammatory disease treatable by the present pharmaceutical composition include, but are not limited to, asthma, allergic rhinitis, acute and chronic liver diseases, atherosclerosis, cancer, Crohn's disease, hypersensitivity lung disease, irritable bowel syndrome (IBS), inflammatory dermatoses, Sjogren's syndrome, systemic inflammatory response syndrome (SIRS), and ulcerative colitis. Examples of the infectious disease treatable by the present pharmaceutical composition include, but are not limited to, bacterial, fungal and viral infections.

In some preferred embodiments, the pharmaceutical composition may further comprise an immunosuppressant, an anti-inflammatory agent or an anti-infectious agent.

The immunosuppressant that may be used with the present pharmaceutical composition is a corticosteroid, a calcineurin inhibitor, a mammalian target of rapamycin (mTOR) inhibitor, an Inosine monophosphate dehydrogenase (IMPDH) inhibitor, a therapeutic protein, or a monoclonal antibody. Examples of corticosteroid suitable as an immunosuppressant include, but are not limited to, prednisone, budesonide, and prednisolone. Examples of calcineurin inhibitor suitable as an immunosuppressant include, but are not limited to, cyclosporine, and tacrolimus. Examples of mTOR inhibitor suitable as an immunosuppressant include, but are not limited to, sirolimus, and everolimus. Examples of IMDH inhibitor suitable as an immunosuppressant include, but are not limited to, azathioprine, leflunomide, and mycophenolate. Examples of therapeutic protein suitable as an immunosuppressant include, but are not limited to, abatacept, adalimumab, anakinra, certolizumab, etanercept, golimumab, infliximab, ixekizumab, natalizumab, rituximab, secukinumab, tocilizumab, ustekinumab, and vedolizumab. Examples of monoclonal antibody suitable as an immunosupressant include, but are not limited to, basiliximab, daclizumab, and muromonab.

The anti-inflammatory agent that may be used with the present pharmaceutical composition is a cyclooxygenase (COX) inhibitor, particularly a COX-2 inhibitor, such as Celebrex, Celecoxib, Rofecoxib, Valdecoxib, DuP-697, Etoricoxib, and Lumiracoxib.

The anti-infectious agent suitable for use in the present pharmaceutical composition may be an anti-biotic that inhibits the growth of Gram-negative or Gram-positive bacteria. Examples of antibiotic suitable as an anti-infectious agent include, but are not limited to, acumycin, ampicillin, amoxycillin, amphotericins, antimycins, anglomycin, avermectins, azithromycin, boromycin, carbomycins, carbapenem, ceftazidime, cethromycin, chloramphenicol, chalcomycin, ciprofloxacin, concanamycins, cirramycin, clarithromycin, colistin, cycloxacillin, daptomycin, desmethyl azithromycin, desertomycins, dihydropikromycin, dirithromycin, doxycycline, enramycin, erythromycin, flurithromycin, flumequin gentamycin, juvenimicins, kujimycins, lankamycins, lincomycin, litorin, leucomycins, megalomicins, meropenem, methymycin, midecamycins, mycinamicin I, mycinamicin II, mycinamicin III, mycinamicin IV, mycinamicin V, mycinamicin VI, mycinamicin VII, mycinamicin VIII, narbomycin, neoantimycin, neomethymycin, netilmicin, neutromycin, niddamycins, norfioxacin, oleandomycins, oligomycins, ossamycin, oxacillin, oxolinic acid, penicillin, pikromycin, piperacillin, platenomycins, rapamycins, relomycin, rifamycins, rosaramicin, roxithromycin, virginiamycin, spiramycin, sporeamycin, staphococcomycin, streptomycin, sulfamethoxazole, swalpamycin, telithromycin, teicoplanin, timentin, tobramycin, ticarcillin, trimethoprim, tetracyclin, zlocillin, and/or a combination thereof.

Certain pharmaceutical compositions are single unit dosage forms suitable for oral, mucosal (e.g., nasal, sublingual, vaginal, buccal, or rectal), parenteral (e.g., subcutaneous, intravenous, bolus injection, intramuscular, or intra-arterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration requires enteric coatings to protect the compounds of this invention from degradation within the gastrointestinal tract. Similarly, a formulation may contain ingredients that facilitate delivery of the active ingredient(s) to the site of action. For example, compounds may be administered in liposomal formulations, in order to protect them from degradative enzymes, facilitate transport in circulatory system, and effect delivery across cell membranes to intracellular sites.

Similarly, poorly soluble compounds may be incorporated into liquid dosage forms (and dosage forms suitable for reconstitution) with the aid of solubilizing agents, emulsifiers and surfactants such as, but not limited to, cyclodextrins (e.g., α-cyclodextrin or β-cyclodextrin), and non-aqueous solvents, such as, but not limited to, ethyl alcohol, isopropyl alcohol, ethyl carbonate, ethyl acetate, benzyl alcohol, benzyl benzoate, propylene glycol, 1,3-butylene glycol, dimethyl formamide, dimethyl sulfoxide (DMSO), biocompatible oils (e.g., cottonseed, groundnut, corn, germ, olive, castor, and sesame oils), glycerol, tetrahydrofurfuryl alcohol, polyethylene glycols, fatty acid esters of sorbitan, and mixtures thereof (e.g., DMSO:corn oil).

The composition, shape, and type of a dosage form will vary depending on its use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art.

### 4.1 Oral Dosage Forms

Pharmaceutical compositions of the present invention suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art.

Typical oral dosage forms are prepared by combining the active ingredient(s) in an intimate admixture with at least one excipient according to conventional pharmaceutical compounding techniques. Excipients can take a wide variety of forms depending on the form of preparation desired for administration.

Because of their ease of administration, tablets and capsules represent the most advantageous oral dosage unit forms. If desired, tablets can be coated by standard aqueous or non-aqueous techniques. Such dosage forms can be prepared by conventional methods of pharmacy. In general, pharmaceutical compositions and dosage forms are prepared by uniformly and intimately admixing the active ingredients with liquid carriers, finely divided solid carriers, or both, and then shaping the product into the desired presentation if necessary. Disintegrants may be incorporated in solid dosage forms to facility rapid dissolution. Lubricants may also be incorporated to facilitate the manufacture of dosage forms (e.g., tablets).

### 4.2 Parenteral Dosage Forms

Parenteral dosage forms can be administered to patients by various routes including, but not limited to, subcutaneous, intravenous (including bolus injection), intramuscular, and intra-arterial. Because their administration typically bypasses patients' natural defenses against contaminants, parenteral dosage forms are specifically sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: water, aqueous vehicles such as, but not limited to, sodium chloride solution, Ringer's solution, and Dextrose; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 4.3 Transdermal, Topical and Mucosal Dosage Forms

Transdermal, topical, and mucosal dosage forms include, but are not limited to, ophthalmic solutions, sprays, aerosols, creams, lotions, ointments, gels, solutions, emulsions, suspensions, or other forms known to one of skill in the art. Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms are well known to those skilled in the pharmaceutical arts, and depend on the particular tissue to which a given pharmaceutical composition or dosage form will be applied.

Depending on the specific tissue to be treated, additional components may be used prior to, in conjunction with, or subsequent to treatment with active ingredients of the invention. For example, penetration enhancers may be used to assist in delivering active ingredients to the tissue.

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates may also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition

### 5. Kits

Also encompasses within the present disclosure is an article of manufacture or “kit,” containing materials useful for the treatment or prophylaxis of a disease and/or disorder mediated by TLR-4 in a subject.

In one embodiment, the kit comprises a container comprising the compound of the present disclosure. The kit is suitable for the treatment or prophylaxis of a disease and/or disorder mediated by TLR-4, such as autoimmune disease, inflammatory disease or infectious disease. Suitable containers include, for example, bottles, vials, syringes, blister pack, and etc. The container may be formed from a variety of materials such as glass, or plastic. The contain may hold a compound of the present disclosure or a pharmaceutical formulation thereof, in an amount effective for the treatment or prophylaxis of the disease and/or disorder mediated by TLR-4, and may have a sterile access port, for example, the container may be an intravenous solution bag or a vail having a stopper pierceable by a hypodermic injection needle). The kit may further comprise a label or package insert on or associated with the container. The label or package insert indicates that the composition is used for treating condition of choice. Alternatively or additionally, the kit may further comprise a second container comprising a pharmaceutically acceptable buffer, such as a phosphate-buffered saline, Ringer's solution or dextrose solution. It may further include other materials desirable from a commercial and user standpoint, including other buffers, diluents, filters, needles, and syringes.

The kit may further include directions for the administration of the compound of the present invention and, if present, the second formulation for treating or preventing the disease and/or disorder mediated by TLR-4. For example, if the kit comprises a first composition comprising the compound of the present disclosure, and a second pharmaceutical formulation, the kit may further include directions for the simultaneous, sequential, or separate administration of the first and second pharmaceutical compositions to a patient in need thereof.

In another embodiment, the kits are suitable for the delivery of solid oral forms of a compound of the present disclosure, such a kit includes, for example, a number of unit dosages. Such kits include card having the dosages oriented in the order of their intended use. An example of such kit is a “blister pack.” Blister packs are well known in the packaging industry and are widely used for packaging pharmaceutical unit dosage forms. If desired, an aid may be provided, for example, in the form of numbers, letters, or other markings or with a calendar insert, designating the days in the treatment schedule in which the dosage can be administered.

According to one embodiment, the Kit may include, at least, (a) a first container containing any of the present compound of Formula (I) to (IV); and optionally, (b) a second container containing a second therapeutic agent that is any of a known TLR-4 antagonist, an anti-inflammatory agent, an anti-biotic, or an immunosuppressant; and (c) a legend associated with the kit for instructing a user how to use the kit. The legend may be in a form of pamphlet, tape, CD, VCD or DVD.

### 6. Method of Use

The present invention encompasses a method for the treatment of a subject having a disease and/or a disorder mediated by TLR-4. The method comprises the step of administering the present pharmaceutical composition, which comprises a therapeutically effective amount of any of the compound of Formula (I) to (IV) of the present disclosure, to the subject, so as to ameliorate, mitigate and/or prevent the symptoms of the disease and/or disorder mediated by TLR-4.

According to embodiments of the present disclosure, the disease and/or disorder mediated by TLR-4 may be an autoimmune disease, an inflammatory disease or an infectious disease. Examples of the autoimmune disease treatable by the present method include, but are not limited to, multiple sclerosis, psoriasis, systemic lupus erythemotosis (SLE), Type I diabetes mellitus, and Wegener's granulomatosis. Examples of the inflammatory disease treatable by the present method include, but are not limited to, asthma, allergic rhinitis, acute and chronic liver diseases, atherosclerosis, cancer, Crohn's disease, hypersensitivity lung disease, irritable bowel syndrome (IBS), inflammatory dermatoses, Sjogren's syndrome, systemic inflammatory response syndrome (SIRS), and ulcerative colitis. Examples of the infectious disease treatable by the present method include, but are not limited to, bacterial, fungal and viral infections.

Accordingly, in some embodiments, the method further includes the step of administering to the subject, an immunosuppressant, an anti-inflammation agent, or an anti-infectious agent before, together with, or after the administration of the present pharmaceutical composition.

The immunosuppressant suitable for use in the present method may be a corticosteroid, a calcineurin inhibitor, a mammalian target of rapamycin (mTOR) inhibitor, an Inosine monophosphate dehydrogenase (IMPDH) inhibitor, a therapeutic protein, or a monoclonal antibody. Examples of corticosteroid suitable as an immunosuppressant include, but are not limited to, prednisone, budesonide, and prednisolone. Examples of calcineurin inhibitor suitable as an immunosuppressant include, but are not limited to, cyclosporine, and tacrolimus. Examples of mTOR inhibitor suitable as an immunosuppressant include, but are not limited to, sirolimus, and everolimus. Examples of IMDH inhibitor suitable as an immunosuppressant include, but are not limited to, azathioprine, leflunomide, and mycophenolate. Examples of therapeutic protein suitable as an immunosuppressant include, but are not limited to, abatacept, adalimumab, anakinra, certolizumab, etanercept, golimumab, infliximab, ixekizumab, natalizumab, rituximab, secukinumab, tocilizumab, ustekinumab, and vedolizumab. Examples of monoclonal antibody suitable as an immunosupressant include, but are not limited to, basiliximab, daclizumab, and muromonab.

The anti-inflammatory agent suitable for use in the present method may be a cyclooxygenase (COX) inhibitor, particularly a COX-2 inhibitor, such as Celebrex, Celecoxib, Rofecoxib, Valdecoxib, DuP-697, Etoricoxib, and Lumiracoxib.

The anti-infectious agent suitable for use in the present method may be an anti-biotic that inhibits the growth of Gram-negative or Gram-positive bacteria. Examples of antibiotic suitable for use as an anti-infectious agent include, but are not limited to, acumycin, ampicillin, amoxycillin, amphotericins, antimycins, anglomycin, avermectins, azithromycin, boromycin, carbomycins, carbapenem, ceftazidime, cethromycin, chloramphenicol, chalcomycin, ciprofloxacin, concanamycins, cirramycin, clarithromycin, colistin, cycloxacillin, daptomycin, desmethyl azithromycin, desertomycins, dihydropikromycin, dirithromycin, doxycycline, enramycin, erythromycin, flurithromycin, flumequin gentamycin, juvenimicins, kujimycins, lankamycins, lincomycin, litorin, leucomycins, megalomicins, meropenem, methymycin, midecamycins, mycinamicin I, mycinamicin II, mycinamicin III, mycinamicin IV, mycinamicin V, mycinamicin VI, mycinamicin VII, mycinamicin VIII, narbomycin, neoantimycin, neomethymycin, netilmicin, neutromycin, niddamycins, norfioxacin, oleandomycins, oligomycins, ossamycin, oxacillin, oxolinic acid, penicillin, pikromycin, piperacillin, platenomycins, rapamycins, relomycin, rifamycins, rosaramicin, roxithromycin, virginiamycin, spiramycin, sporeamycin, staphococcomycin, streptomycin, sulfamethoxazole, swalpamycin, telithromycin, teicoplanin, timentin, tobramycin, ticarcillin, trimethoprim, tetracyclin, zlocillin, and/or a combination thereof.

The amount, route of administration and dosing schedule of the present pharmaceutical composition will depend upon factors such as the specific indication to be treated, prevented, or managed, and the age, sex and condition of the patient. The roles played by such factors are well known in the art, and may be accommodated by routine experimentation.

The present invention will now be described more specifically with reference to the following embodiments, which are provided for the purpose of demonstration rather than limitation. While they are typically of those that might be used, other procedures, methodologies, or techniques known to those skilled in the art may alternatively be used.

### Examples

Materials and Methods

Cell Culture

The HEK293 cells were cultured in the manufacturer's suggested medium supplemented with 10% heat-inactivated fetal bovine serum (FBS), 100 units/mL penicillin, and 100 g/mL streptomycin, at 37° C. in a humidified 5% CO₂ incubator.

Cell Viability Assay

HEK 293 cells (2.5-5.0×10⁴ cells/well) were seeded in 96-well plate for overnight and treated with different compounds for 16-24 hours. Cell growth was determined by MTh-based colorimetric assays. Viability of the test cells treated with vehicle (0.5% DMSO) only was defined as 100% viable. Survival of cells after treatment with DETDs was calculated using the following formula:

Viable cell number (%)=OD₅₇₀ (treated cell culture)/OD₅₇₀ (vehicle control)×100.

### Example 1 Preparation of the Compound of Formula (I)

### 1.1 Synthesis of Compound 3

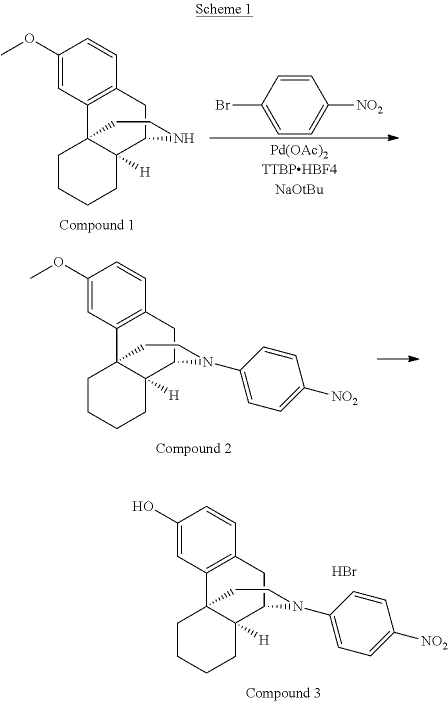 [SMILES: COc1ccc2c(c1)[C@]13CCCC[C@@H]1[C@H](C2)NCC3 ; O=[N+]([O-])c1ccc(Br)cc1 ; COc1ccc2c(c1)[C@]13CCCC[C@@H]1[C@H](C2)N(c1ccc([N+](=O)[O-])cc1)CC3 ; O=[N+]([O-])c1ccc(N2CC[C@@]34CCCC[C@@H]3[C@@H]2Cc2ccc(O)cc24)cc1 ; Br]

Substrate solution: 3-methoxymorphinan (Compound 1) (520 mg, 2.02 mmol) and 1-bromo-4-nitrobenzene (493 mg, 2.44 mmol) in toluene (3 ml).

Catalyst solution: TTBP.HBF₄ (Tri-tert-butylphosphonium tetrafluoroborate) (10.4 mg, 0.04 mol) and NaO^{t}Bu (289.9 mg, 3.02 mmol) were mixed in toluene (2 ml) and stirred for 5 minutes, then Pd(OAc)₂ (6 mg, 0.03 mmol) in toluene (1 ml) was added by syringe, stirred under room temperature for 30 minutes.

Compound 3 was produced by the process of Scheme 1, in which a substrate solution containing Compound 1 (520 mg, 2.02 mmol) was added to a catalyst solution by syringe, and the mixture was heated to reflux under Ar (g) for 29 hours. After that, the reaction mixture was diluted with 50 ml ethyl acetate (EA) and washed with dH₂O (50 ml) and brine. Organic portion was separated and adsorbed on silica dust, and purified on Merck grade 9385 silica. Eluent system was 0-20% EA/n-hexane. Fraction with R_{f}=3.9 (20% EA/n-hexane) were collected to give Compound 2 as an orange-yellow solid (779 mg, yield is 10%).

To a stirred solution of Compound 2 (0.100 mg, 0.26 mmol) were added HBr (3.0 mL) at room temperature. The reaction mixture was stirred at 110° C. for 4 hours. After cooling to room temperature, the resulting mixture was poured into H₂O, filtered to afford the product (Compound 3) as yellow solid (72 mg, yield was 62%).

Spectral data of Compound 3:

¹H-NMR (300 MHz, DMSO): δ 8.03 (d, 2H), 7.02 (d, 2H), 6.85 (d, 1H), 6.74 (d, 1H), 6.54 (dd, 1H), 4.29 (s, 1H), 3.64 (dd, 1H), 2.97 (dd, 1H), 2.57-2.42 (m, 2H), 2.33 (d, 1H), 1.78-1.00 (m, 10H). ESI-MS m/z calcd for C₂₂H₂₅BrN₂O₃ 444.10, found 365.18 (No HBr) [M+H]⁺.

### 1.2 Synthesis of Compound 4

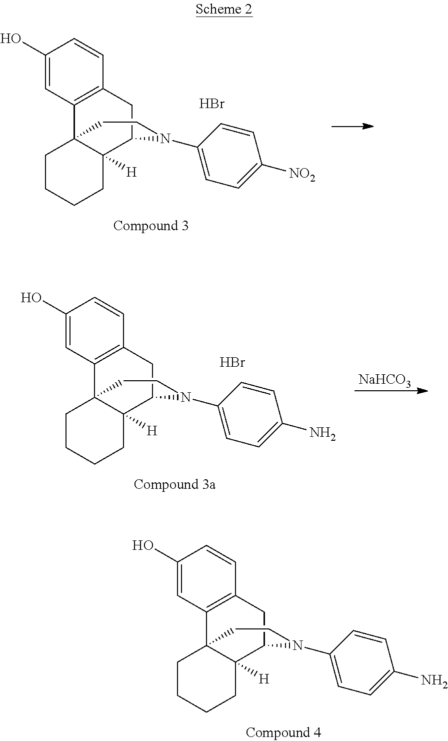

To a solution of Compound 3 (0.142 g, 0.39 mmol) in Methanol (4.0 mL), was added 5% Pd/C (0.114 g, 0.05 mmol). Hydrogenation was carried out under a pressure of 1 atm. After stirring for overnight, the catalyst was filtered through a pad of celite, the filtrate was concentrated with rotary evaporator to get the Compound 3a. Compound 3a was then added into aqueous NaHCO₃, stirred for 3 hours and extracted with ethyl acetate (2×30 mL). The combined organic phases were washed with water, dried over sodium sulfate and concentrated. The residue was purified by column chromatography on silica gel (50% ethyl acetate/hexane) and obtained Compound 4. Yield of Compound 4 was 26% (34 mg). Black solids.

Spectral Data of Compound 4:

¹H-NMR (300 MHz, CDCl₃): δ 6.89-6.84 (m, 2H), 6.78 (s, 1H), 6.77-6.74 (m, 2H), 6.55 (dd, 1H), 3.77 (d, 1H), 3.02 (dd, 1H), 2.66-2.58 (m, 3H), 2.33 (d, 1H), 2.00 (dt, 1H), 1.80 (td, 1H), 1.68 (d, 1H), 1.55-1.46 (m, 3H), 1.43-1.29 (m, 3H), 1.14 (qd, 2H). ESI-MS m/z calcd for C₂₂H₂₆N₂O 334.20, found 335.21 [M+H]⁺.

### 1.3 Synthesis of Compound 5

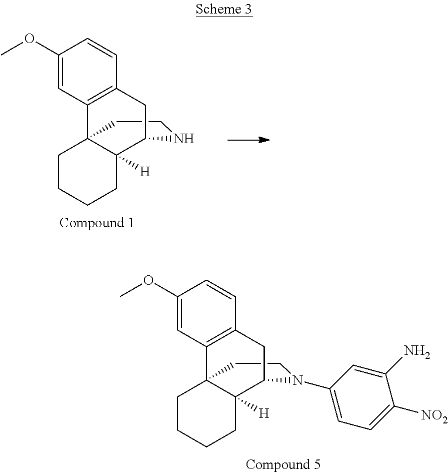 [SMILES: COc1ccc2c(c1)[C@]13CCCC[C@@H]1[C@H](C2)NCC3 ; COc1ccc2c(c1)[C@]13CCCC[C@@H]1[C@H](C2)N(c1ccc([N+](=O)[O-])c(N)c1)CC3]

Compound 5 was synthesized by following the process of scheme 1, using the starting material of 5-bromo-2-nitroaniline. Yield of Compound 5 was 22% (37 mg). Red solids.

Spectral Data of Compound 5:

¹H-NMR (300 MHz, CDCl₃): δ 8.00 (d, 1H), 6.94 (d, 1H), 6.88 (d, 1H), 6.70 (dd, 1H), 6.34 (dd, 1H), 6.15 (s, 2H), 5.90 (d, 1H), 4.08 (s, 1H), 3.80 (s, 3H), 3.46 (dd, 1H) 3.05 (dd, 1H), 2.72-2.60 (m, 2H) 2.41 (d, 1H), 1.84 (dt, 1H), 1.80-1.70 (m, 2H), 1.60-1.51 (m, 3H), 1.43-1.26 (m, 3H), 1.13-1.10 (m, 1H). ESI-MS m/z calcd for C₂₃H₂₇N₃O₃ 393.21, found 394.21 [M+H]⁺.

### 1.3a Synthesis of Compound 6

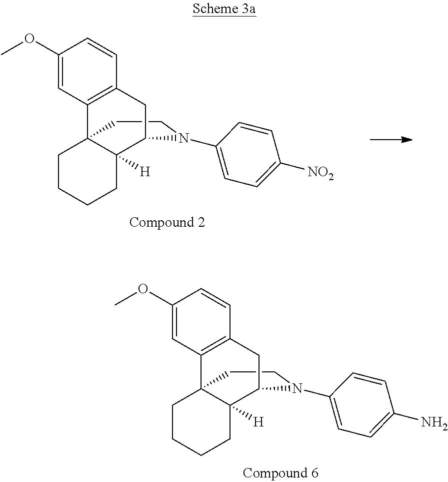 [SMILES: COc1ccc2c(c1)[C@]13CCCC[C@@H]1[C@H](C2)N(c1ccc([N+](=O)[O-])cc1)CC3 ; COc1ccc2c(c1)[C@]13CCCC[C@@H]1[C@H](C2)N(c1ccc(N)cc1)CC3]

To a solution of compound 2 (0.70 g, 1.85 mmol) in ethanol (12.0 mL) and EA (15 mL), was added 5% Pd/C (0.56 g) and CH₃CO₂H (6 mL). Hydrogenation was carried out under a pressure of 1 atm. After stirring for overnight, the catalyst was filtered through a pad of celite, the filtrate was concentrated with rotary evaporator. The crude compound 6 was purified by column chromatography on silica gel (ethyl acetate/hexane=1:8) and was obtained 127 mg.

Spectral Data of Compound 6:

¹H-NMR (300 MHz, CDCl3): δ 6.93-6.90 (d, 1H), 6.85-6.84 (d, 1H), 6.79-6.74 (m, 2H), 6.69-6.60 (m, 3H), 3.78 (s, 3H), 3.76-3.73 (m, 1H), 3.05 (br, 2H), 2.99-2.94 (m, 1H), 2.68-2.66 (d, 2H), 2.63-2.54 (td, 1H), 2.42-2.38 (m, 1H), 2.01-1.95 (m, 1H), 1.86-1.76 (td, 1H), 1.69-1.65 (m, 1H), 1.56-1.35 (m, 6H), 1.19-1.10 (m, 1H). ESI-MS m/z calcd for C₂₃H₂₈N₂O 348.22, found 349.2 [M+H]⁺

### 1.4 Synthesis of Compound 7

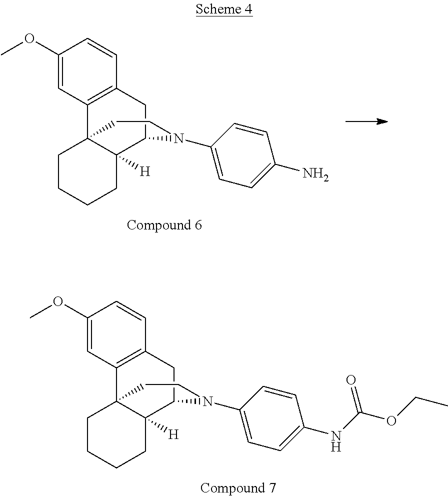 [SMILES: COc1ccc2c(c1)[C@]13CCCC[C@@H]1[C@H](C2)N(c1ccc(N)cc1)CC3 ; CCOC(=O)Nc1ccc(N2CC[C@@]34CCCC[C@@H]3[C@@H]2Cc2ccc(OC)cc24)cc1]

To a stirred solution of Compound 6 (0.127 mg, 0.36 mmol) in tetrahydrofuran (THF) (1.5 mL), N,N-diisopropylethylamine (DIPEA) (0.094 mL, 0.54 mmol) and Ethyl chloroformate (0.041 mL, 0.43 mmol) at 0° C. were added. The reaction mixture was stirred at room temperature for overnight. After cooling to room temperature, the resulting mixture was concentrated, diluted in dicholomethane (DCM), poured into H₂O, and extracted with DCM three times. The combined organic extracts were washed with brine, dried over anhydrous MgSO₄, filtered, and concentrated under reduced pressure. The residue was purified by flash column chromatography on silica gel (Hexanes-EtOAc=14:86) to afford the product (Compound 7). Yield: (57 mg, 0.14 mmol, 38%), white solid.

Spectral Data of Compound 7:

¹H-NMR (300 MHz, CDCl₃): δ 7.15 (d, 2H), 6.94 (d, 1H), 6.86-6.84 (m, 3H), 6.69 (dd, 1H), 4.13 (d, 1H), 3.90 (s, 1H), 3.80 (s, 3H), 3.22 (dd, 1H), 2.88-2.81 (m, 1H), 2.74-2.68 (m, 1H), 2.62 (td, 1H), 2.44-2.40 (m, 1H), 1.93 (dt, 1H) 1.80 (td, 1H), 1.72-1.68 (m, 1H), 1.58-1.50 (m, 3H), 1.48-1.30 (m, 3H), 1.28-1.08 (m, 6H). ESI-MS m/z calcd for C₂₆H₃₂N₂O₃ 420.24, found 421.55 [M+H]⁺.

### 1.5 Synthesis of Compound 8

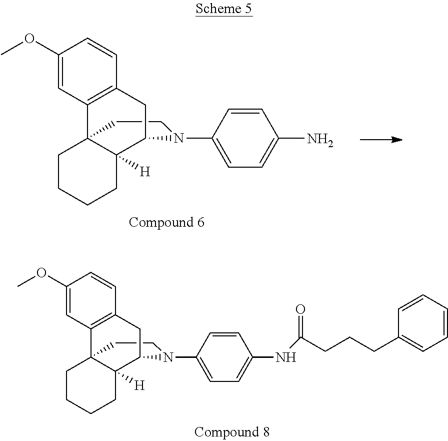 [SMILES: COc1ccc2c(c1)[C@]13CCCC[C@@H]1[C@H](C2)N(c1ccc(N)cc1)CC3 ; COc1ccc2c(c1)[C@]13CCCC[C@@H]1[C@H](C2)N(c1ccc(NC(=O)CCCc2ccccc2)cc1)CC3]

To a solution of 4-phenylbutanoic acid (41.2 mg, 0.25 mmol) in fresh, dried DCM (2 ml) in an ice water bath, was added TEA (37 μl, 0.27 mmol) and ethyl chloroformate (25 μl, 0.26 mmol) dropwise. Reaction mixture was stirred in an ice water bath for 1 hour and compound 6 (89.7 mg, 0.26 mmol) in DCM (1.5 ml) was added dropwise. Reaction mixture was kept stirring from 0° C. to room temperature overnight dH₂O (5 ml) was added to the reaction mixture. Organic portion was separated and washed with sat. NaHCO₃ and sat. brine sequentially. After being dried over anhydrous MgSO₄, solvent was evaporated on a rotavapor. The crude was purified further on a silica column to give 93.3 mg of product (Compound 8)(white solids), and the yield thereof was 78%.

Spectral data of Compound 8:

¹H-NMR (300 MHz, CDCl₃): δ 7.35 (d, 2H), 7.30-7.15 (m, 3H), 7.19 (d, 2H), 7.03 (s, 1H), 6.93-6.80 (m, 4H), 6.66 (d, 1H), 3.97 (s, 1H), 3.76 (s, 3H), 3.10 (d, 1H), 2.80-2.57 (m, 3H), 2.40 (d, 1H), 2.32-2.25 (m, 2H), 2.11-1.95 (m, 2H), 1.88-1.10 (m, 12H). ESI-MS m/z calcd for C₃₃H₃₈N₂O₂ 494.29, found 495.30 [M+H]⁺.

### 1.6 Synthesis of Compound 9

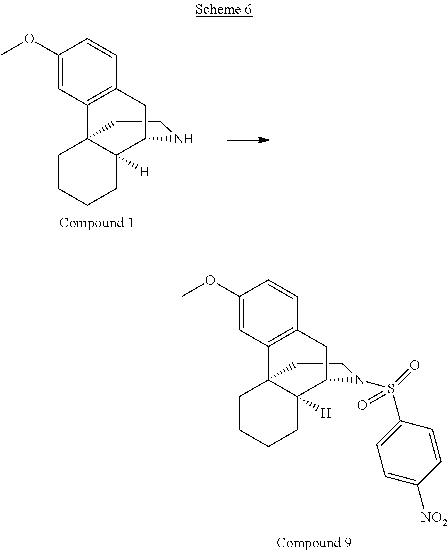 [SMILES: COc1ccc2c(c1)[C@]13CCCC[C@@H]1[C@H](C2)NCC3 ; COc1ccc2c(c1)[C@]13CCCC[C@@H]1[C@H](C2)N(S(=O)(=O)c1ccc([N+](=O)[O-])cc1)CC3]

To a solution of Compound 1 (0.100 mg, 0.39 mmol) in DCM (1.5 mL) were added Et₃N (0.082 mL, 0.59 mmol) and 4-nitrobenzenesufonyl chloride (104.2 mg, 0.47 mmol) at 0° C. The reaction mixture was stirred at room temperature for overnight. After cooling to room temperature, the resulting mixture was poured into H₂O, and extracted with DCM three times. The combined organic extracts were washed with brine, dried over anhydrous MgSO₄, filtered, and concentrated under reduced pressure. The residue was purified by flash column chromatography on silica gel (DCM-EtOAc=17:83) to afford the product (Compound 9) as a yellow solid. Yield was 73% (63 mg).

Spectral data of Compound 9:

¹H-NMR (300 MHz, CDCl₃): δ 8.34 (d, 2H), 8.00 (d, 2H), 6.86 (d, 1H), 4.14 (s, 1H), 3.75 (s, 3H), 3.60 (dd, 1H), 3.00 (dd, 1H), 2.74 (td, 1H), 2.44 (d, 1H), 2.40-2.30 (m, 1H), 1.74-1.00 (m, 10H). ESI-MS m/z calcd for C₂₃H₂₆N₂O₅S 442.16, found 443.16 [M+H]⁺.

### 1.7 Synthesis of Compound 10

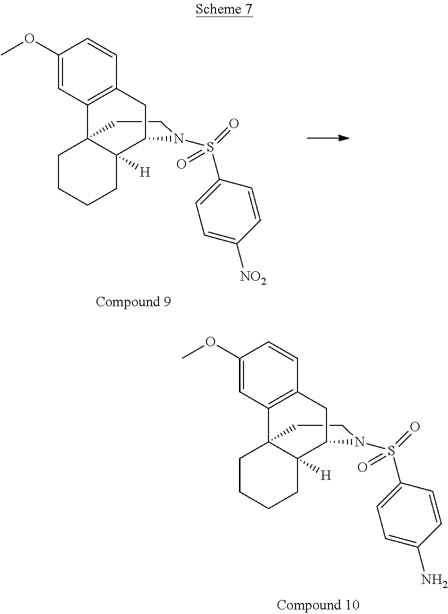 [SMILES: COc1ccc2c(c1)[C@]13CCCC[C@@H]1[C@H](C2)N(S(=O)(=O)c1ccc([N+](=O)[O-])cc1)CC3 ; COc1ccc2c(c1)[C@]13CCCC[C@@H]1[C@H](C2)N(S(=O)(=O)c1ccc(N)cc1)CC3]

Compound 10 was synthesized in accordance with the process set forth in Scheme 2, using compound 9 as the starting material. Yield of Compound 10 was 80% (194 mg). White solids.

Spectral data of Compound 10:

¹H-NMR (300 MHz, CDCl₃): δ 7.56 (d, 2H), 6.85 (d, 1H), 6.77 (d, 1H), 6.69-6.64 (m, 3H), 4.20 (s, 2H), 4.10-4.05 (m, 1H), 3.73 (s, 3H), 3.53 (dd, 1H), 2.88 (dd, 1H), 2.64 (td, 1H), 2.52 (d, 1H), 2.34-2.28 (m, 1H), 1.78-1.70 (m, 1H), 1.66-1.56 (m, 2H), 1.50-1.40 (m, 2H), 1.34-1.20 (m, 3H), 1.06-0.8 (m, 2H). ESI-MS m/z calcd for C₂₃H₂₈N₂O₃S 412.18, found 413.19 [M+H]⁺.

### 1.8 Synthesis of Compound 11

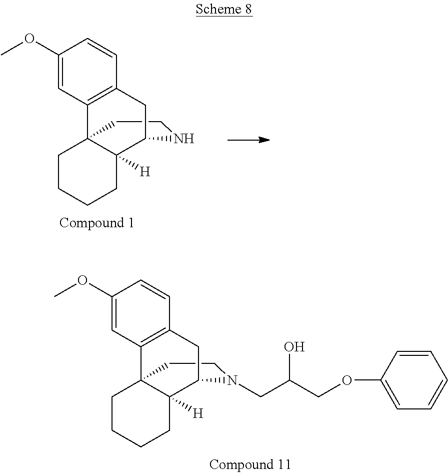 [SMILES: COc1ccc2c(c1)[C@]13CCCC[C@@H]1[C@H](C2)NCC3 ; COc1ccc2c(c1)[C@]13CCCC[C@@H]1[C@H](C2)N(CC(O)COc1ccccc1)CC3]

Compound 1 (76.24 mg in 0.8 ml 95% EtOH) was reacted with 2-(phenoxymethyl)oxirane (40 mg, 0.27 mmol) in 95% EtOH (2 ml) under inert atmosphere at reflux for 22 hours (oil bath: 90° C.). To the residue was added DCM (10 ml) and sat. NaHCO₃ (aq) (10 ml). Aqueous layer was extracted with DCM (5 ml each, twice). Organic portions were combined, washed with sat, brine, dried over anhydrous MgSO₄ and evaporated. The crude was purified on silica column (Merck grade 9385) using 30%-50% EA/n-hexane with 2% TEA as eluent. Fraction with R_{f} 0.59 (50% EA/n-hexane, 3 drops TEA) were collected to give the title compound (Compound 11) as a yellow liquid (yield: 25.2 mg, 23%).

Spectral Data of Compound 11:

¹H-NMR (400 MHz, CDCl₃): δ 7.30-7.24 (m, 2H), 7.04 (d, 1H), 6.96-6.90 (m, 3H), 6.80 (d, 1H), 6.70 (d, 1H), 4.06-3.95 (m, 3H), 3.77 (s, 3H), 2.97-2.70 (m, 4H), 2.64-2.52 (m, 1H), 2.47-2.40 (m, 2H), 2.37-2.30 (m, 1H), 1.80-1.60 (m, 3H), 1.56-1.50 (m, 1H), 1.42-1.23 (m, 5H), 1.15-0.8 (m, 2H). ESI-MS m/z calcd for C₂₆H₃₃NO₃ 407.25, found 408.25 [M+H]⁺.

### 1.9 Synthesis of Compound 12

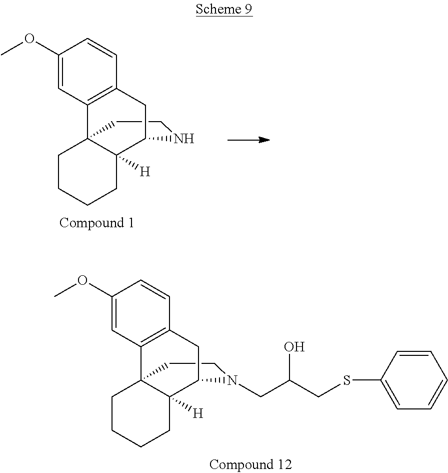 [SMILES: COc1ccc2c(c1)[C@]13CCCC[C@@H]1[C@H](C2)NCC3 ; COc1ccc2c(c1)[C@]13CCCC[C@@H]1[C@H](C2)N(CC(O)CSc1ccccc1)CC3]

Compound 12 was synthesized in accordance with the method set forth in Scheme 8 by use of the starting material of 2-((phenylthiol)methyl)oxirane. Yield of Compound 12 was 21% (44.7 mg).

Spectral Data of Compound 12:

¹H-NMR (400 MHz, CDCl₃): δ 7.34 (d, 2H), 7.27-7.20 (m, 2H), 7.19-7.10 (m, 1H), 7.00 (d, 1H), 6.77 (s, 1H), 6.70 (d, 1H), 4.56 (s, 1H), 3.80 (s, 1H), 3.75 (s, 3H), 3.15-3.07 (m, 1H), 3.00-2.90 (m, 1H), 2.97-2.70 (m, 3H), 2.55-2.28 (m, 4H), 2.10 (t, 1H), 1.95 (d, 1H), 1.50-1.24 (m, 9H), 1.11-1.03 (m, 1H). ESI-MS m/z calcd for C₂₆H₃₃NO₂S 423.22, found 424.23 [M+H]⁺.

### 1.10 Synthesis of Compound 13

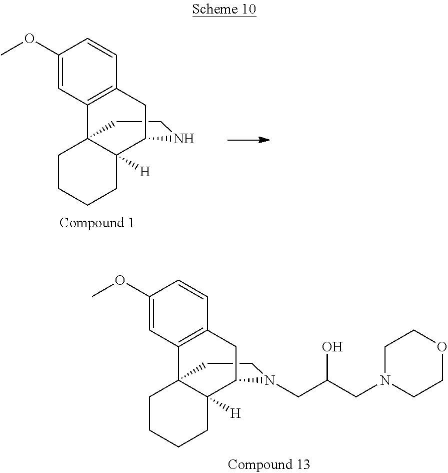 [SMILES: COc1ccc2c(c1)[C@]13CCCC[C@@H]1[C@H](C2)NCC3 ; COc1ccc2c(c1)[C@]13CCCC[C@@H]1[C@H](C2)N(CC(O)CN1CCOCC1)CC3]

Compound 13 was synthesized in accordance with the method set forth in Scheme 8 by use of the starting material of 4-(oxiran-2-ylmethyl)morpholine. Yield of Compound 13 was 78% (176 mg). Yellow liquid.

Spectral Data of Compound 13:

¹H-NMR (400 MHz, CDCl₃): δ 6.93 (d, 1H), 6.70 (s, 1H), 6.60 (d, 1H), 3.78-3.70 (m, 1H), 3.69 (s, 3H), 3.64 (s, 3H), 2.85-2.71 (m, 2H), 2.70-2.58 (m, 2H), 2.50-2.39 (m, 5H), 2.37-2.15 (m, 5H), 2.00 (t, 1H), 1.80-1.52 (m, 4H), 1.46-1.40 (m, 1H), 1.30-1.17 (m, 3H), 1.05-0.95 (m, 2H). ESI-MS m/z calcd for C₂₄H₃₆N₂O₃ 400.27, found 401.28 [M+H]⁺.

### 1.11 Synthesis of Compound 15

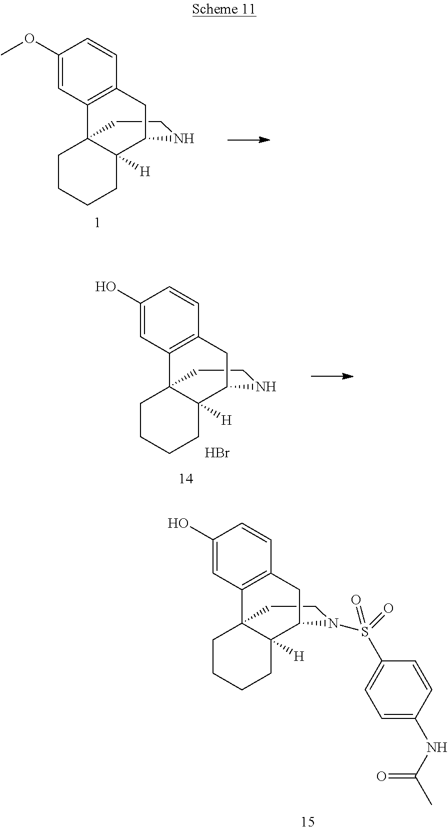 [SMILES: COc1ccc2c(c1)[C@]13CCCC[C@@H]1[C@H](C2)NCC3 ; Oc1ccc2c(c1)[C@]13CCCC[C@@H]1[C@H](C2)NCC3 ; Br ; CC(=O)Nc1ccc(S(=O)(=O)N2CC[C@@]34CCCC[C@@H]3[C@@H]2Cc2ccc(O)cc24)cc1]

Compound 1 (2.0 g, 7.77 mmol) was dissolved in 47% HBr (60 mL). It was heated at 110° C. for 6 h. It was cooled down to rt and was diluted with water (30 mL) and treated with c-NH₄OH until pH=8. It was extracted with chloroform (30 mL). The organic layer was pumped down to dryness to give solid Compound 14.

To a solution of Compound 14 (0.5 g, 1.54 mmol) in dry CH₂Cl₂ (10 mL) were added triethylamine (322 μL, 2.31 mmol) and N-acetylsulfonyl chloride (0.43 g, 1.85 mmol) sequentially and the mixture was stirred for 16 h at room temperature. To the mixture was added sat aq NaCl solution (2 mL) and the mixture was extracted with EtOAc (3 mL×3). The combined organic layer was dried over anhydrous MgSO₄ filtered, and concentrated. Column chromatography of the crude product gave 196 mg (29%) of a white solid of Compound 15.

Spectra Data of Compound 15:

¹H-NMR (400 MHz, CDCl3): δ 7.86 (s, 1H), 7.73 (d, 2H), 7.67 (d, 2H), 7.66 (m, 2H), 6.81-6.78 (m, 2H), 6.70-6.67 (dd, 1H), 4.11-4.08 (m, 1H), 3.57-3.53 (dd, 1H), 2.91-2.85 (dd, 1H), 2.49-2.42 (m, 2H), 2.23 (s, 3H), 2.08-2.05 (m, 1H), 1.72-1.54 (m, 4H), 1.43-1.40 (m, 1H), 1.28-1.17 (m, 2H), 0.96-0.87 (m, 2H). ESI-MS m/z calcd for C₂₄H₂₈N₂O₄S 440.18, found 441.56 [M+H]⁺.

### 1.12 Synthesis of Compound 16

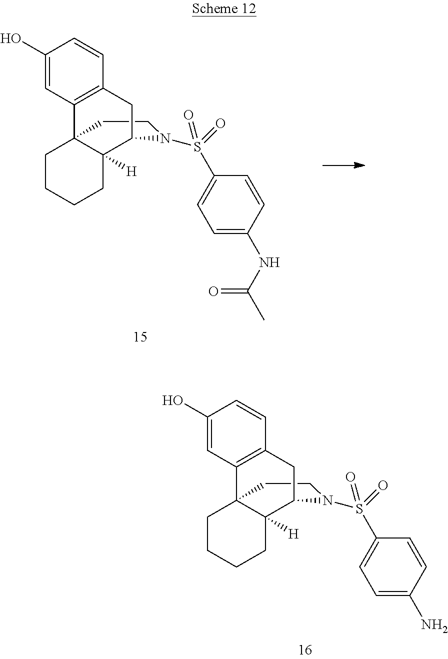 [SMILES: CC(=O)Nc1ccc(S(=O)(=O)N2CC[C@@]34CCCC[C@@H]3[C@@H]2Cc2ccc(O)cc24)cc1 ; Nc1ccc(S(=O)(=O)N2CC[C@@]34CCCC[C@@H]3[C@@H]2Cc2ccc(O)cc24)cc1]

Compound 15 was dissolved in EtOH 4 mL was added 12N hydrochloric acid (1.5 mL) and heated at 100° C. for 5 h. The solution is neutralized at 0° C. with NaOH(aq) and the mixture was extracted with EtOAc (3 mL×3). The combined organic layer was dried over anhydrous MgSO₄ filtered, and concentrated. Column chromatography of the crude product gave 24 mg (55%) of a white solid of Compound 16.

Spectra Data of Compound 16:

¹H-NMR (400 MHz, CD3OD): S 7.48-7.46 (d, 2H), 7.35-7.33 (d, 2H), 6.92-6.85 (m, 2H), 6.72-6.70 (d, 2H), 6.60-6.59 (m, 2H), 6.57 (m, 1H), 3.97 (m, 1H), 2.92-2.86 (dd, 1H), 2.53-2.38 (m, 2H), 2.05-2.01 (d, 1H), 1.66-1.58 (m, 2H), 1.56-1.45 (m, 1H), 1.44-1.37 (m, 2H), 1.35-1.20 (m, 1H), 1.15-1.08 (m, 2H), 1.00-0.86 (m, 2H).

### 1.13 Synthesis of Compounds 17 and 18

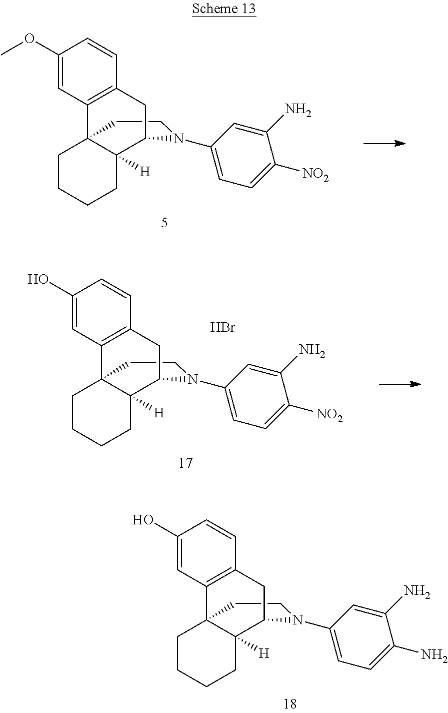 [SMILES: COc1ccc2c(c1)[C@]13CCCC[C@@H]1[C@H](C2)N(c1ccc([N+](=O)[O-])c(N)c1)CC3 ; Nc1cc(N2CC[C@@]34CCCC[C@@H]3[C@@H]2Cc2ccc(O)cc24)ccc1[N+](=O)[O-] ; Br ; Nc1ccc(N2CC[C@@]34CCCC[C@@H]3[C@@H]2Cc2ccc(O)cc24)cc1N]

The synthetic methods for Compound 17 and 18 follow the Scheme 1 and 2, respectively.

Spectra data of Compound 17:

¹H-NMR (400 MHz, DMSO): δ 9.05 (br, 1H), 7.79 (d, 1H), 6.87 (d, 1H), 6.72 (d, 1H), 6.56-6.53 (dd, 1H), 6.46-6.42 (dd, 1H), 6.20 (d, 1H), 5.4-4.9 (br, 2H), 4.11 (s, 1H), 3.46-3.37 (m, 1H), 2.95-2.89 (dd, 1H), 2.44 (m, 1H) 2.34 (d, 1H), 1.73-1.70 (m, 1H), 1.69-1.53 (m, 2H), 1.52-1.39 (m, 3H), 1.39-1.21 (m, 2H), 1.21-1.11 (m, 2H), 1.04-0.98 (m, 1H). ESI-MS m/z calcd for C₂₂H₂₆BrN₃O₃ 459.12, found 380.4[M+H]⁺ (free base).

Spectra Data of Compound 18:

¹H-NMR (400 MHz, CD3OD): δ 8.01 (s, 1H), 7.49 (d, 1H), 7.10 (dd, 1H), 7.06 (s, 1H), 6.82-6.78 (m, 2H), 6.57-6.54 (dd, 1H), 4.94 (m, 4H), 3.96 (m, 1H), 3.13-3.05 (dd, 1H), 2.77-2.67 (m, 2H), 2.61-2.57 (m, 1H), 2.46-2.43 (m, 1H), 2.03-1.99 (m, 1H), 1.88-1.80 (m, 1H), 1.73-1.70 (m, 1H), 1.56-1.50 (m, 2H), 1.49-1.25 (m, 3H), 1.21-1.17 (dd, 1H), 0.99-0.90 (m, 1H).

### 1.14 Synthesis of Compounds 19 and 20

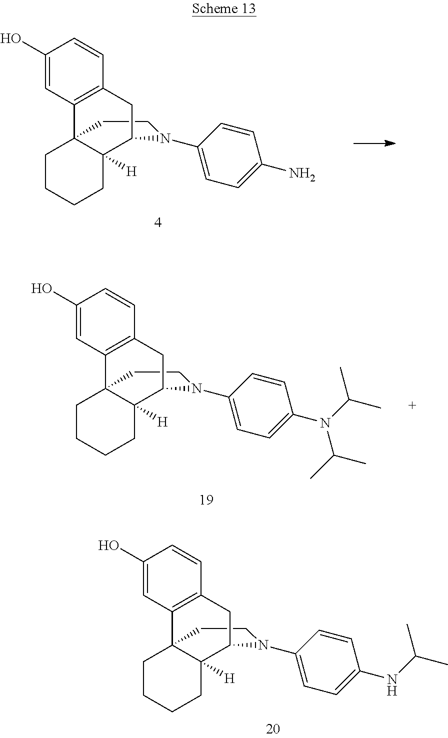 [SMILES: Nc1ccc(N2CC[C@@]34CCCC[C@@H]3[C@@H]2Cc2ccc(O)cc24)cc1 ; CC(C)N(c1ccc(N2CC[C@@]34CCCC[C@@H]3[C@@H]2Cc2ccc(O)cc24)cc1)C(C)C ; CC(C)Nc1ccc(N2CC[C@@]34CCCC[C@@H]3[C@@H]2Cc2ccc(O)cc24)cc1]

To a solution of the Compound 4 (100 mg, 0.299 mmol) in MeOH (6 mL), acetone (1.5 mL), acetic acid (51 μL) and NaHB(OAc)₃ (59 mg, 0.89 mmol) were added. The reaction mixture was stirred at room temperature for 16 hours. The resulting mixture was poured into aqueous NaHCO₃, stirred for 3 hours and extracted with dichloromethane (2×30 mL). The combined organic phases were washed with water, dried over sodium sulfate and concentrated. The residue was purified by column chromatography on silica gel (50% ethyl acetate/hexane) and obtained Compound 19 and 20, separately.

Spectra Data of Compound 19:

¹H-NMR (400 MHz, CDCl3): δ 6.96-6.94 (d, 2H), 6.88-6.86 (d, 1H), 6.81-6.77 (m, 3H), 6.60-6.57 (dd, 1H), 3.14-3.10 (dd, 1H), 2.78-2.63 (m, 2H), 2.62-2.56 (m, 1H), 2.35-2.32 (d, 1H), 1.97-1.93 (m, 1H), 1.82-1.76 (m, 1H), 1.72-1.65 (m, 1H), 1.56-1.46 (m, 3H), 1.44-1.30 (m, 4H), 1.27-1.12 (m, 4H), 1.05 (s, 6H), 0.97 (s, 6H). ESI-MS m/z calcd for C₂₈H₃₈N₂O 418.63, found 419.5 [M+H]⁺.

Spectra Data of Compound 20:

¹H-NMR (400 MHz, CDCl₃): δ 6.89-6.76 (m, 4H), 6.58-6.54 (m, 3H), 3.78 (m, 1H), 3.57-3.53 (m, 1H), 2.98-2.94 (dd, 1H), 2.63-2.56 (m, 1H), 2.36-2.33 (d, 1H), 2.00-1.97 (m, 1H), 1.84-1.77 (m, 1H), 1.69-1.66 (m, 2H), 1.56-1.46 (m, 4H), 1.44-1.32 (4H), 1.26-1.21 (m, 2H), 1.19 (s, 3H), 1.18 (s, 3H). ESI-MS m/z calcd for C₂₅H₃₂N₂O 376.54, found 377.5[M+H]⁺.

### 1.15 Synthesis of Compound 21

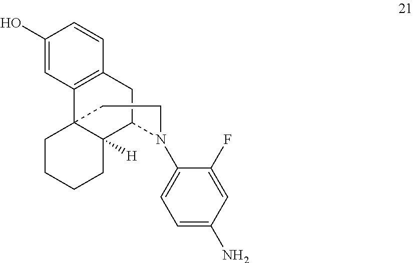 [SMILES: Nc1ccc(N2CC[C@@]34CCCC[C@@H]3[C@@H]2Cc2ccc(O)cc24)c(F)c1]

Compound 21 was synthesized by using 1-bromo-2-fluoro-4-nitrobenzene and following Scheme 1 and 2 accordingly.

Spectra Data of Compound 21:

¹H-NMR (400 MHz, CDCl3): δ 6.91 (d, 1H), 6.78 (m, 1H), 6.74-6.70 (m, 1H), 6.63-6.60 (dd, 1H), 6.46-6.42 (dd, 1H), 6.39-6.36 (dd, 1H), 4.63 (s, 1H), 3.51-3.48 (m, 2H), 2.85-2.79 (m, 1H), 2.70-2.66 (m, 1H), 2.63-2.62 (m, 2H), 2.36-2.32 (m, 1H), 2.06 (t, 1H), 1.90-1.82 (m, 1H), 1.67-1.60 (m, 1H), 1.55-1.33 (m, 7H), 1.13-1.1.04 (m, 1H). ESI-MS m/z calcd for C₂₂H₂₅FN₂O 352.20, found 353.4[M+H]⁺.

### 1.16 Synthesis of Compound 22

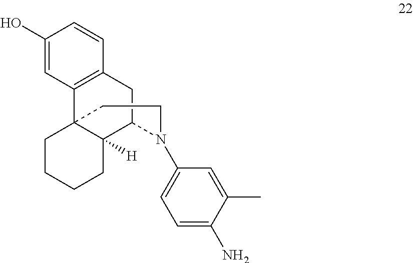 [SMILES: Cc1cc(N2CC[C@@]34CCCC[C@@H]3[C@@H]2Cc2ccc(O)cc24)ccc1N]

Compound 22 was synthesized by using 1-bromo-3-methyl-4-nitrobenzene and following Schemes 1 and 2 accordingly.

Spectra Data of Compound 22:

¹H-NMR (300 MHz, CDCl₃): δ 6.88-6.85 (d, 1H), 6.78 (d, 1H), 6.69-6.56 (m, 4H), 4.74 (s, 1H), 3.74 (m, 1H), 3.33 (m, 2H), 2.98-2.95 (m, 1H), 2.66-2.56 (m, 3H), 2.37-2.33 (m, 1H), 2.15 (s, 3H), 2.05-1.97 (m, 1H), 1.84-1.77 (m, 1H), 1.66-1.42 (m, 4H), 1.38-1.35 (m, 2H), 1.28-1.26 (m, 1H), 1.23-1.08 (m, 1H). ESI-MS m/z calcd for C₂₃H₂₈N₂O 348.22, found 349.4[M+H]⁺.

### 1.17 Synthesis of Compound 23

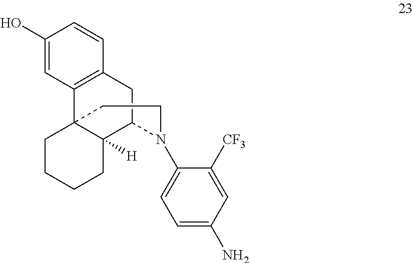 [SMILES: Nc1ccc(N2CC[C@@]34CCCC[C@@H]3[C@@H]2Cc2ccc(O)cc24)c(C(F)(F)F)c1]

Compound 23 was synthesized by using 1-bromo-2-trifluoromethyl-4-nitrobenzene and following Schemes 1 and 2 accordingly.

Spectra Data of Compound 23:

¹H-NMR (400 MHz, CDCl₃): δ 7.10-7.08 (d, 1H) 7.0-6.98 (d, 1H), 6.95-6.94 (d, 1H), 6.79-6.76 (m, 2H), 6.65-6.62 (dd, 1H), 4.66 (s, 1H) 3.72-3.60 (br, 2H), 3.06 (m, 1H), 2.98-2.87 (m, 2H), 2.61-2.55 (dd, 1H), 2.53-2.50 (m, 1H), 2.34-2.30 (m, 1H), 2.07-2.03 (m, 2H), 1.84-1.78 (td, 1H), 1.47-1.29 (m, 6H), 1.24-1.05 (m, 1H). ESI-MS m/z calcd for C₂₃H₂₅F₃N₂O 402.19, found 403.4[M+H]⁺.

### 1.18 Synthesis of Compound 24

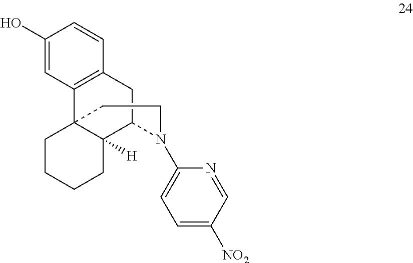 [SMILES: O=[N+]([O-])c1ccc(N2CC[C@@]34CCCC[C@@H]3[C@@H]2Cc2ccc(O)cc24)nc1]

Compound 24 was synthesized by using 2-bromo-5-nitropyridine and following Scheme 1 accordingly.

Spectra Data of Compound 24:

¹H-NMR (400 MHz, CDCl₃): δ 9.05 (d, 1H), 8.19-8.16 (dd, 1H), 6.99-6.95 (d, 1H), 6.83 (d, 1H), 6.66-6.63 (dd, 1H), 6.56-6.53 (d, 1H), 4.72 (s, 1H), 3.76-3.73 (m, 1H), 3.20-3.14 (dd, 1H), 2.80 (m, 1H), 2.65-2.60 (d, 1H), 2.43-2.2.38 (m, 1H), 1.87-1.68 (m, 4H), 1.61-1.52 (m, 4H), 1.41-1.17 (m, 3H). ESI-MS m/z calcd for C₂₁H₂₃N₃O₃ 365.17, found 366.3[M+H]⁺.

### 1.19 Synthesis of Compound 25

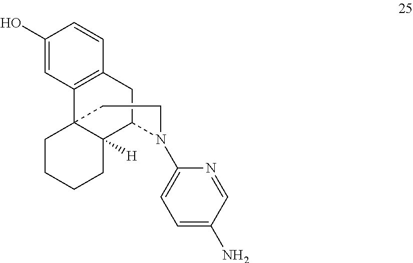 [SMILES: Nc1ccc(N2CC[C@@]34CCCC[C@@H]3[C@@H]2Cc2ccc(O)cc24)nc1]

Compound 25 was synthesized by using Compound 24 and following Scheme 2 accordingly.

Spectra Data of Compound 25:

¹H-NMR (400 MHz, CD3OD): S 7.72-7.71 (d, 1H), 7.13-7.10 (dd, 1H), 6.84-6.81 (d, 1H), 6.78-6.77 (d, 1H), 6.68-6.66 (d, 1H), 6.57-6.54 (dd, 1H), 4.61 (br, 2H), 4.31-4.28 (m, 1H), 3.43-3.39 (m, 1H), 2.90-2.86 (dd, 1H), 2.62-2.54 (td, 1H), 2.52-2.48 (d, 1H), 2.44-2.41 (m, 1H), 1.88-1.82 (m, 1H), 1.77-1.68 (m, 2H), 1.58-1.28 (m, 7H), 1.24-1.1.17 (m, 1H). ESI-MS m/z calcd for C₂₁H₂₅N₃O 335.2, found 336.4[M+H]⁺.

### Example 2 Characterization of Compounds of Example 1

### 2.1 Effects of the Compound of Example 1 on TLR4 Activity and Cell Viability

Stimulation of the Toll-like receptor 4 (TLR4) was determined by assessing activation of the transcription factor NF-κB in HEK293 cells that were engineered to express TLR4. Assessment of TLR4 stimulation was based on the use of an NF-κB-inducible secreted alkaline phosphatase (SEAP) reporter system in which the SEAP reporter was under the control of a promoter inducible by NF-κB. Thus, the degree of activation of TLR4 can be indirectly quantified spectrophotometrically by measuring the amount of the SEAP reporter that is produced.

The TLR4-expressing cells were plated in HEK-Blue Detection (Invivogen, San Diego, Calif.) medium in a 96-well plate (25,000-50,000 cells/well). The cells were stimulated with lipopolysaccharides (LPS) alone or in combination with the compound of example 1. Cell culture without LPS and the compound of example 1 was defined as baseline control. To test whether the compound of example 1 could block that activation of the TLR4, the cells were treated with the compound of example 1 (20 μM) and LPS (final conc. 10 ng/ml/well). After a16-24 hours incubation period at 37° C. in a CO₂ incubator, The signals of individual samples in 96-well plates were detected at O.D.₆₅₀ nm with a Molecular Devices SpectraMax i3 Imaging Cytometer. The TLR4 activity was expressed in percentage of LPS alone-stimulated cells minus baseline control.

The ell viability assay was performed by the MTT method. Cells were incubated with 45 μl of 5 mg/ml MTT for 1 hour at 37° C., and 150 μl of DMSO was added at the end of culture to dissolve the crystals. The signals of individual samples in 96-well plates were detected at O.D._{570 nm} with Molecular Device SpectraMax Imaging Cytometer. Cell viability was expressed in percentage of viable LPS-stimulated cells. Results are summarized in Table 3.

TABLE 3
Effects of the compounds of example 1
on TLR-4 activity and cell viability

| | |
|---|---|
| 20 μM | 20 μM |
| TLR-4 inhibition (%) | Cell viability (%) |

| Compound | Average | SD | Average | SD |
|---|---|---|---|---|
| 3 | 0.0 | 2.2 | 82.1 | 3.0 |
| 4 | 90.5 | 1.1 | 92.2 | 2.8 |
| 5 | 13.2 | 0.1 | 110.9 | 2.1 |
| 6 | 37.7 | 2.4 | 83.1 | 2.0 |
| 7 | 0.0 | 1.9 | 79.3 | 1.0 |
| 8 | 6.0 | 1.6 | 104.7 | 4.2 |
| 9 | 23.7 | 1.1 | 104.1 | 3.9 |
| 10 | 30.0 | 4.1 | 111.6 | 4.8 |
| 11 | 54.5 | 0.4 | 88.8 | 0.3 |
| 12 | 59.1 | 0.6 | 81.3 | 0.5 |
| 13 | 2.8 | 0.9 | 101.5 | 5.5 |
| 15 | 45.1 | 1.3 | 104.0 | 1.1 |
| 16 | 42.1 | 0.1 | 75.5 | 1.7 |
| 17 | 53.1 | 1.8 | 116.0 | 3.7 |
| 18 | 57.7 | 1.3 | 143.0 | 5.5 |
| 19 | 75.3 | 0.8 | 99.2 | 2.6 |
| 20 | 99.8 | 0.2 | 89.9 | 0.3 |
| 21 | 13.8 | 0.8 | 97.4 | 1.2 |
| 22 | 64.2 | 2.6 | 101.7 | 1.4 |
| 23 | 22.9 | 0.1 | 96.2 | 1.5 |
| 24 | 43.5 | 0.3 | 103.3 | 1.8 |
| 25 | 13.3 | 0.7 | 100.8 | 0.6 |

It is evident from Table 3 that compounds 4 and 20 are strong antagonists of TLR-4, compounds 11, 12, 17, 18, 19, and 22 show remarkable inhibitions on TLR-4, and compounds 6, 9, 10, 15, 16, and 24 respectively possess moderate to mild inhibitory activity toward TLR-4; whereas compounds 3, 5, 7, 8, 13, 21, 23, and 25 have weak to negligible activity towards TLR-4. Further, each compound of example 1 exhibited none or minor cytotoxicity to the tested cells.

It will be apparent to those skilled in the art that various modifications and variations can be made to the disclosed embodiments. It is intended that the specification and examples be considered as exemplary only, with a true scope of the disclosure being indicated by the following claims and their equivalents.

1.-17. (canceled)

18. A compound of Formula (I): 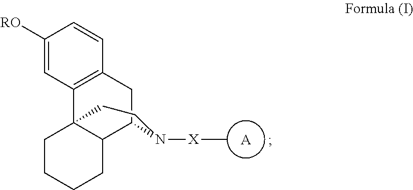
wherein:
R is H, C₁₋₂₀ alkyl, aryl, —(CH₂)ₙ—OR₁, or —(CH₂)ₙ—NR₁R₂;
X is nil, —SO₂—, or 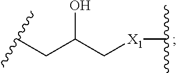
A is cycloalkyl, heterocycloalkyl, phenyl, aryl or heteroaryl;
n is an integral between 1 to 10;
R₁ and R₂ are independently H, C₁₋₂₀ alkyl, C₁₋₂₀ alkoxy, phenyl, or aryl;
X₁ is nil, S or O;
each C₁₋₂₀ alkyl and C₁₋₂₀ alkoxy is optionally substituted with at least one substituent selected from the group consisting of halogen, hydroxy, alkoxy, and phenyl;
each cycolalkyl, heterocycloalkyl, phenyl, aryl and heteroaryl is optionally substituted with at least one substituent selected from the group consisting of halogen, C₁₋₂₀ alkyl, hydroxyl, alkoxy, haloalkyl, haloalkoxy, nitro, —NR₃R₄, —(CO)R₁ and —(CO)OR₁; and
R₃ and R₄ are independently H, C₁₋₂₀ alkyl, phenyl, aryl, —(CO)R₁ or —(CO)OR₁; and
in the case when X₁ is nil, then A is not 4-nitrophenyl, 4-hydroxylphenyl, or 4-methoxyphenyl;
or a pharmaceutically acceptable salt, solvate, hydrate, co-crystal, and stereoisomer thereof.

19. The compound as claimed in claim 18, wherein the compound is a toll-like receptor 4 (TLR-4) antagonist.

20. The compound as claimed in claim 18, wherein the compound of Formula (I) has the structure of Formula (II): 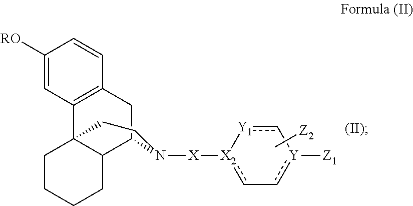
wherein:
R is independently H, C₁₋₂₀ alkyl, aryl, —(CH₂)ₙ—OR₁, or —(CH₂)ₙ—NR₁R₂;
X is nil, —SO₂—, or 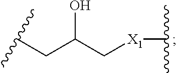 [symbol]
is a single or double bond;
X₁ is nil, S or O;
X₂ is N or C;
Y is N, C or O;
Y₁ is C or N;
n is an integral between 1 to 10;
Z₁ and Z₂ are independently H, halogen, C₁₋₂₀ alkyl, —NR₁R₂, nitro, —OR′, —NH(CO)R₁, and —N(CO)OR₁;
R₁ and R₂ are independently H, halogen, C₁₋₂₀ alkyl, C₁₋₂₀ alkoxy, phenyl, or aryl;
each C₁₋₂₀ alkyl and C₁₋₂₀ alkoxy is optionally substituted with at least one substituent selected from the group consisting of halogen, hydroxy, alkoxy, and phenyl;
each phenyl and aryl is optionally substituted with at least one substituent selected from the group consisting of halogen, C₁₋₂₀ alkyl, hydroxyl, alkoxy, haloalkoxy, nitro, —NR₃R₄, —(CO)R₁ and —(CO)OR₁; and
R₃ and R₄ are independently H, C₁₋₂₀ alkyl, phenyl, aryl, —(CO)R₁ or —(CO)OR₁; and
in the case when X is nil, X₂ and Y are independently C, [symbol] is double bond, then Z₁ is not nitro, methoxy, or hydroxyl.

21. The compound as claimed in claim 18, wherein the compound of Formula (I) has the structure of Formula (III): 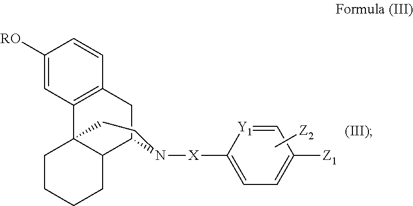
wherein:
R is independently H, or C₁₋₂₀ alkyl;
X is nil or —SO₂—;
Y₁ is C or N;
Z₁ and Z₂ are independently H, —NR₁R₂, nitro, —OR, —R₅, or —NH(CO)R₅; and
R₁ and R₂ are independently H, halogen, C₁₋₂₀ alkyl, C₁₋₂₀ alkoxy, phenyl, or aryl;
R₅ is H, halogen, C₁₋₂₀ alkyl or C₁₋₂₀ alkoxy optionally substituted with halogen or phenyl; and
in the case when X is nil, then Z₁ is not nitro, hydroxyl, or methoxy.

22. The compound as claimed in claim 21, wherein R is H, X is nil, Z₁ is amino, and Z₂ is H.

23. The compound as claimed in claim 21, wherein R is methyl, X is SO₂, Z₂ is H, and Z₂ is amino.

24. The compound as claimed in claim 18, wherein the compound of Formula (I) has the structure of Formula (IV): 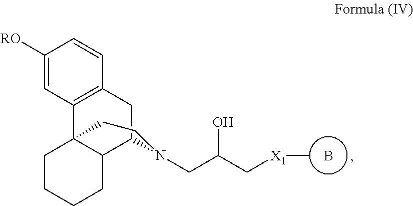
wherein:
R is independently H, or C₁₋₂₀ alkyl;
X₁ is nil, S or O;
B is phenyl or heterocycloalkyl optionally substituted with halogen, C₁₋₂₀ alkyl, alkoxy, haloalkoxy, amino or nitro.

25. The compound as claimed in claim 24, wherein R is methyl, X₁ is S or O, and B is phenyl.

26. The compounds selected from the compounds delineated in Table A or its geometric isomers, enantiomers, diastereomers, racemates, pharmaceutically acceptable salts, prodrugs and solvates thereof:
TABLE A

| Compound Number | Structure |
|---|---|
| 3 | 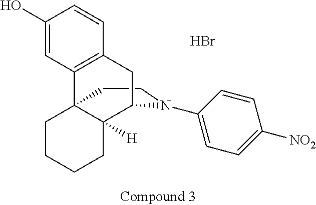 [SMILES: O=[N+]([O-])c1ccc(N2CC[C@@]34CCCC[C@@H]3[C@@H]2Cc2ccc(O)cc24)cc1 ; Br] |
| 4 | 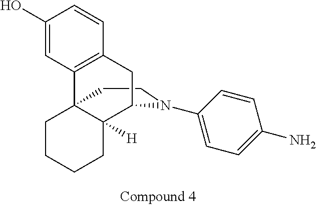 [SMILES: Nc1ccc(N2CC[C@@]34CCCC[C@@H]3[C@@H]2Cc2ccc(O)cc24)cc1] |
| 5 | 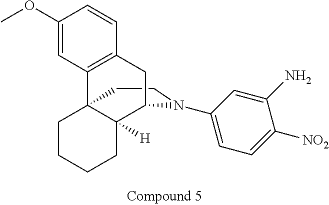 [SMILES: COc1ccc2c(c1)[C@]13CCCC[C@@H]1[C@H](C2)N(c1ccc([N+](=O)[O-])c(N)c1)CC3] |
| 6 | 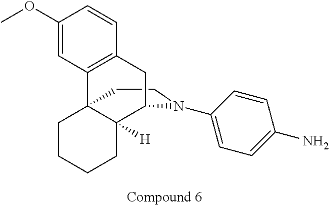 [SMILES: COc1ccc2c(c1)[C@]13CCCC[C@@H]1[C@H](C2)N(c1ccc(N)cc1)CC3] |
| 7 | 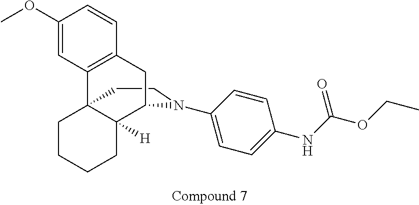 [SMILES: CCOC(=O)Nc1ccc(N2CC[C@@]34CCCC[C@@H]3[C@@H]2Cc2ccc(OC)cc24)cc1] |
| 8 | 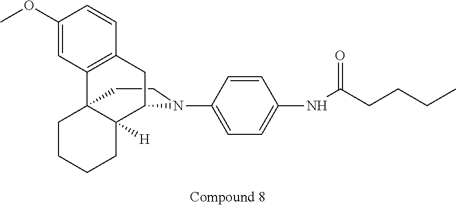 [SMILES: CCCCC(=O)Nc1ccc(N2CC[C@@]34CCCC[C@@H]3[C@@H]2Cc2ccc(OC)cc24)cc1] |
| 9 | 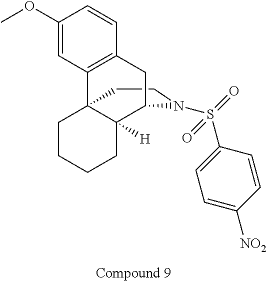 [SMILES: COc1ccc2c(c1)[C@]13CCCC[C@@H]1[C@H](C2)N(S(=O)(=O)c1ccc([N+](=O)[O-])cc1)CC3] |
| 10 | 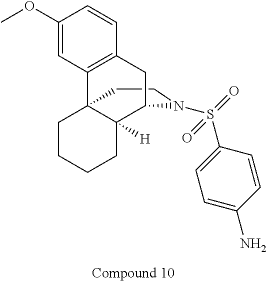 [SMILES: COc1ccc2c(c1)[C@]13CCCC[C@@H]1[C@H](C2)N(S(=O)(=O)c1ccc(N)cc1)CC3] |
| 11 | 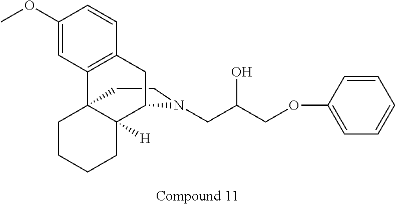 [SMILES: COc1ccc2c(c1)[C@]13CCCC[C@@H]1[C@H](C2)N(CC(O)COc1ccccc1)CC3] |
| 12 | 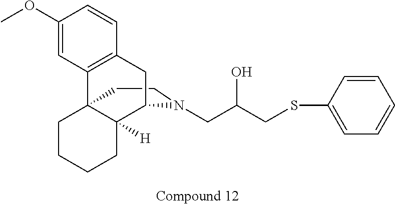 [SMILES: COc1ccc2c(c1)[C@]13CCCC[C@@H]1[C@H](C2)N(CC(O)CSc1ccccc1)CC3] |
| 13 | 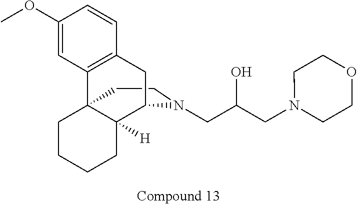 [SMILES: COc1ccc2c(c1)[C@]13CCCC[C@@H]1[C@H](C2)N(CC(O)CN1CCOCC1)CC3] |
| 15 | 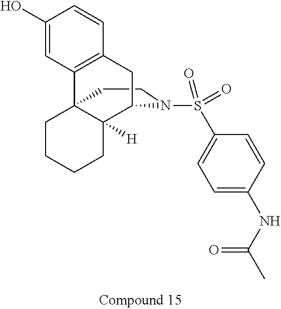 [SMILES: CC(=O)Nc1ccc(S(=O)(=O)N2CC[C@@]34CCCC[C@@H]3[C@@H]2Cc2ccc(O)cc24)cc1] |
| 16 | 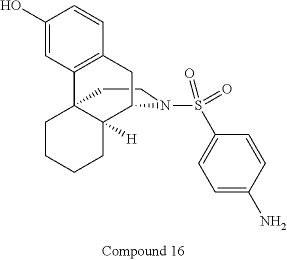 [SMILES: Nc1ccc(S(=O)(=O)N2CC[C@@]34CCCC[C@@H]3[C@@H]2Cc2ccc(O)cc24)cc1] |
| 17 | 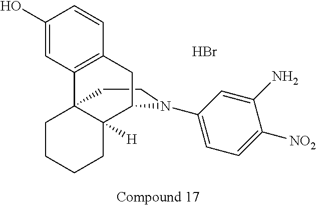 [SMILES: Nc1cc(N2CC[C@@]34CCCC[C@@H]3[C@@H]2Cc2ccc(O)cc24)ccc1[N+](=O)[O-] ; Br] |
| 18 | 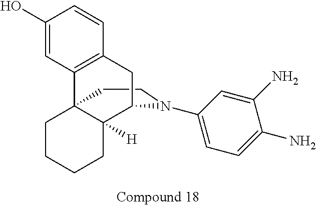 [SMILES: Nc1ccc(N2CC[C@@]34CCCC[C@@H]3[C@@H]2Cc2ccc(O)cc24)cc1N] |
| 19 | 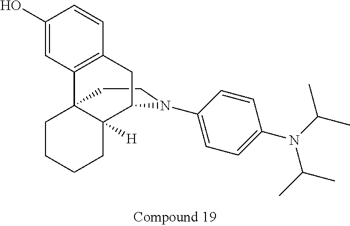 [SMILES: CC(C)N(c1ccc(N2CC[C@@]34CCCC[C@@H]3[C@@H]2Cc2ccc(O)cc24)cc1)C(C)C] |
| 20 | 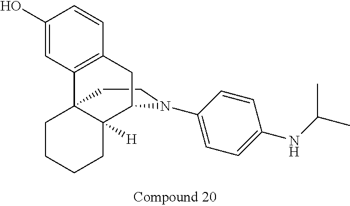 [SMILES: CC(C)Nc1ccc(N2CC[C@@]34CCCC[C@@H]3[C@@H]2Cc2ccc(O)cc24)cc1] |
| 21 | 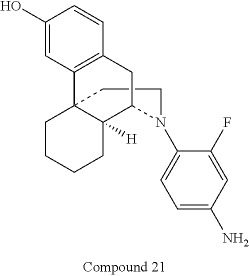 [SMILES: Nc1ccc(N2CC[C@@]34CCCC[C@@H]3[C@@H]2Cc2ccc(O)cc24)c(F)c1] |
| 22 | 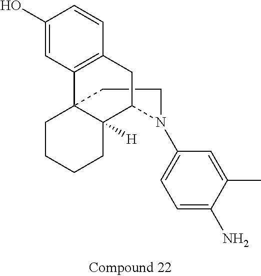 [SMILES: Cc1cc(N2CC[C@@]34CCCC[C@@H]3[C@@H]2Cc2ccc(O)cc24)ccc1N] |
| 23 | 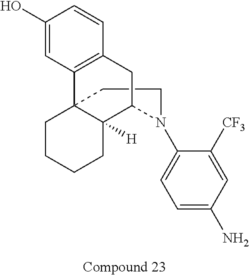 [SMILES: Nc1ccc(N2CC[C@@]34CCCC[C@@H]3[C@@H]2Cc2ccc(O)cc24)c(C(F)(F)F)c1] |
| 24 | 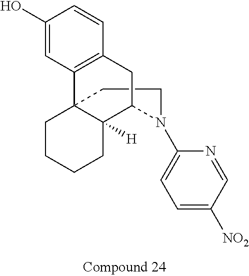 [SMILES: O=[N+]([O-])c1ccc(N2CC[C@@]34CCCC[C@@H]3[C@@H]2Cc2ccc(O)cc24)nc1] |
| 25 | 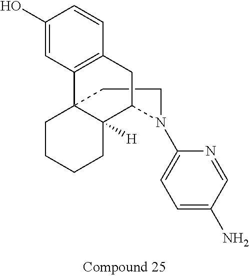 [SMILES: Nc1ccc(N2CC[C@@]34CCCC[C@@H]3[C@@H]2Cc2ccc(O)cc24)nc1] |

27. A pharmaceutical composition for the treatment of a subject having or suspected of having a disease and/or disorder mediated by toll-like receptor 4 (TLR-4) comprising an effective amount of the compound of claim 18, an enantiomer, a diastereoisomer, or a pharmaceutically acceptable salt thereof; and a pharmaceutically acceptable carrier.

28. The pharmaceutical composition as claimed in claim 27, wherein the disease and/or disorder mediated by TLR-4 is an autoimmune disease, or an inflammatory disease.

29. The pharmaceutical composition as claimed in claim 28, wherein the autoimmune disease is selected from the group consisting of multiple sclerosis, psoriasis, systemic lupus erythematosus (SLE), Type I diabetes mellitus, and Wegener's granulomatosis.

30. The pharmaceutical composition as claimed in claim 28, wherein the inflammatory disease is selected from the group consisting of asthma, allergic rhinitis, acute and chronic liver diseases, atherosclerosis, cancer, Crohn's disease, hypersensitivity lung disease, irritable bowel syndrome (IBS), inflammatory dermatoses, Sjogren's syndrome, Systemic inflammatory response syndrome (SIRS), and ulcerative colitis.

31. A method of treating a subject having or suspected of having a disease and/or disorder mediated by toll-like receptor 4 (TLR-4) comprising administering to the subject the pharmaceutical composition of claim 27, so as to ameliorate or alleviate symptoms associated with the disease and/or disorder mediated by TLR-4.

32. The method as claimed in claim 31, wherein the disease and/or disorder mediated by TLR-4 is an autoimmune disease, or an inflammatory disease.

33. The method as claimed in claim 32, wherein the autoimmune disease is selected from the group consisting of multiple sclerosis, psoriasis, systemic lupus erythematosus (SLE), Type I diabetes mellitus, and Wegener's granulomatosis.

34. The method as claimed in claim 32, wherein the inflammatory disease is selected from the group consisting of asthma, allergic rhinitis, acute and chronic liver diseases, atherosclerosis, cancer, Crohn's disease, hypersensitivity lung disease, irritable bowel syndrome (IBS), inflammatory dermatoses, Sjogren's syndrome, systemic inflammatory response syndrome (SIRS), and ulcerative colitis.
